(19) United States
(12) Patent Application Publication
(10) Pub. No.: US 2006/0211707 A1
(43) Pub. Date: Sep. 21, 2006

(54) Piperazine-alkyl-ureido derivatives

(76) Inventors: Hamed Aissaoui, Pulversheim (FR); Christoph Binkert, Basel (CH); Martine Clozel, Winterhalde (CH); Boris Mathys, Egerkingen (CH); Claus Mueller, Weil am Rhein (DE); Oliver Nayler, Arlesheim (CH); Michael Scherz, Ettingen (CH); Jorg Velker, Huningue (FR); Thomas Weller, Binningen (CH)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/555,429

(22) PCT Filed: May 4, 2004
(86) PCT No.: PCT/EP04/04716

§ 371 Date: Nov. 3, 2005

(30) Foreign Application Priority Data
May 7, 2003 (WO) PCT/EP03/04774

(51) Int. Cl. A61K 31/496; C07D 403/02

(52) U.S. Cl. 514/253.06; 514/253.01; 544/360; 544/363

(57) ABSTRACT

The invention relates to novel piperazine derivatives and related compounds and their use as active ingredients in the preparation of pharmaceutical compositions. The invention also concerns related aspects including processes for the preparation of the compounds, pharmaceutical compositions containing one or more of those compounds and especially their use as neurohormonal antagonists.

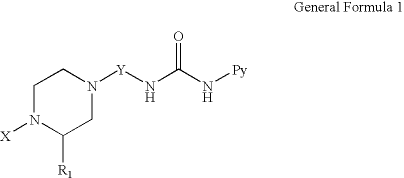

### FIELD OF THE INVENTION

The present invention relates to novel 4-(piperazinyl-alkyl-ureido)-quinoline derivatives of the general formula 1 and their use as active ingredients in the preparation of pharmaceutical compositions. The invention also concerns related aspects including processes for the preparation of the compounds, pharmaceutical compositions containing one or more compounds of the general formula 1 and especially their use as neurohormonal antagonists.

### BACKGROUND OF THE INVENTION

Urotensin II is a cyclic 11-amino acid peptide neurohormone considered to be the most potent vasoconstrictor known, up to 28-fold more potent than endothelin-1. The effects of urotensin II are mediated through activation of a G-protein coupled receptor, the UT receptor, also known as GPR14 or SENR (Ames R S, et al, “Human urotensin-II is a potent vasoconstrictor and agonist for the orphan receptor GPR14” Nature (1999) 401, 282-6. Mori M, Sugo T, Abe M, Shimomura Y, Kurihara M, Kitada C, Kikuchi K, Shintani Y. Kurokawa T, Onda H, Nishimura O, Fujino M. “Urotensin II is the endogenous ligand of a G-protein-coupled orphan receptor, SENR (GPR14)” Biochem. Biophys. Res. Commun. (1999) 265,123-9. Liu Q, Pong S S, Zeng Z, et al, “Identification of urotensin II as the endogenous ligand for the orphan G-protein-coupled receptor GPR14” Biochem. Biophys. Res. Commun. (1999) 266, 174-178.) Urotensin II and its receptor are conserved across evolutionarily distant species, suggesting an important physiological role for the system (Bern H A, Pearson D, Larson B A, Nishioka R S. “Neurohormones from fish tails: the caudal neurosecretory system. I. Urophysiology and the caudal neurosecretory system of fishes” Recent Prog. Horm. Res. (1985) 41, 533-552). In euryhaline fish, urotensin II has an osmoregulatory role, and in mammals urotensin II exerts potent and complex hemodynamic actions. The response to urotensin II is dependent on the anatomical source and species of the tissue being studied. (Douglas S A, Sulpizio A C, Piercy V, Sarau H M, Ames R S, Alyar N V, Ohlstein E H, Willette R N. “Differential vasoconstrictor activity of human urotensin-II in vascular tissue isolated from the rat, mouse, dog, pig, marmoset and cynomolgus monkey” Br. J. Pharmacol. (2000) 131, 1262-1274. Douglas, S A, Ashton D J, Sauermelch C F, Coatney R W, Ohlstein D H, Ruffolo M R, Ohlstein E H, Alyar N V, Willette R “Human urotensin-II is a potent vasoactive peptide: pharmacological characterization in the rat, mouse, dog and primate” J. Cardiovasc. Pharmacol. (2000) 36, Suppl 1:S163-6).

Like other neurohormones, urotensin II has growth stimulating and profibrotic actions in addition to its vasoactive properties. Urotensin II increases smooth muscle cell proliferation, and stimulates collagen synthesis (Tzandis A, et al, “Urotensin II stimulates collagen synthesis by cardiac fibroblasts and hypertrophic signaling in cardiomyocytes via G(alpha)q- and Ras-dependent pathways” J. Am. Coll. Cardiol. (2001) 37, 164A. Zou Y, Nagai R, and Yamazaki T, “Urotensin II induces hypertrophic responses in cultured cardiomyocytes from neonatal rats” FEBS Lett (2001) 508, 57-60). Urotensin II regulates hormone release (Silvestre R A, et al, “Inhibition of insulin release by urotensin II-a study on the perfused rat pancreas” Horm Metab Res (2001) 33, 379-81). Urotensin II has direct actions on atrial and ventricular myocytes (Russell F D, Molenaar P, and O'Brien D M “Cardiostimulant effects of urotensin-II in human heart in vitro” Br. J. Pharmacol. (2001) 132, 5-9). Urotensin II is produced by cancer cell lines and its receptor is also expressed in these cells. (Takahashi K, et al, “Expression of urotensin II and urotensin II receptor mRNAs in various human tumor cell lines and secretion of urotensin II-like immunoreactivity by SW-13 adrenocortical carcinoma cells” Peptides (2001) 22, 1175-9; Takahashi K, et al, “Expression of urotensin II and its receptor in adrenal tumors and stimulation of proliferation of cultured tumor cells by urotensin II” Peptides (2003) 24, 301-306; Shenouda S, et al, “Localization of urotensin-II immunoreactivity in normal human kidneys and renal carcinoma” J Histochem Cytochem (2002) 50, 885-889). Urotensin II and its receptor are found in spinal cord and brain tissue, and intracerebroventricular infusion of urotensin II into mice induces behavioral changes (Gartlon J, et al, “Central effects of urotensin-II following ICV administration in rats” Psychopharmacology (Berlin) (2001) 155, 426-33).

Dysregulation of urotensin II is associated with human disease. Elevated circulating levels of urotensin II are detected in hypertensive patients, in heart failure patients, in diabetic patients, and in patients awaiting kidney transplantation (Totsune K, et al, “Role of urotensin II in patients on dialysis” Lancet (2001) 358, 810-1; Totsune K, et al, “Increased plasma urotensin II levels in patients with diabetes mellitus” Clin Sci (2003) 104, 1-5; Heller J, et al, “Increased urotensin II plasma levels in patients with cirrhosis and portal hypertension” J Hepatol (2002) 37, 767-772).

Substances with the ability to block the actions of urotensin II are expected to prove useful in the treatment of various diseases. WO-2001/45694, WO-2002/78641, WO-2002/78707, WO-2002/79155, WO-2002/79188, WO-2002/89740, WO-2002/89785, WO-2002/89792, WO-2002/89793, WO-2002/90337, WO-2002/90348 and WO-2002/90353 disclose certain sulfonamides as urotensin II receptor antagonists, and their use to treat diseases associated with a urotensin II imbalance. WO-2001/45700 and WO-2001/45711 disclose certain pyrrolidines or piperidines as urotensin II receptor antagonists and their use to treat diseases associated with a urotensin II imbalance. WO-2002/047456 and WO-2002/47687 disclose certain 2-amino-quinolones as urotensin II receptor antagonists and their use to treat diseases associated with a urotensin II imbalance. WO-2002/058702 discloses certain 2-amino-quinolines as urotensin II receptor antagonists and their use to treat diseases associated with a urotensin II imbalance. These derivatives are different from the compounds of the present invention as they do not bear a substituted urea function in the 4-position of the quinoline ring. WO-2001/66143 discloses certain 2,3-dihydro-1H-pyrrolo[2,3-b]quinolin-4-ylamine derivatives useful as urotensin II receptor antagonists, WO-2002/00606 discloses certain biphenyl compounds useful as urotensin II receptor antagonists, and WO-2002/02530 discloses certain piperazines useful as urotensin II receptor antagonists. These compounds are different from the compounds of the present invention as they do not comprise urea derivatives bearing a 4-pyridinyl-like moiety. WO-02/076979 and WO-03/048154 disclose certain quinoline derivatives as urotensin II receptor antagonists, and their use to treat diseases associated with a urotensin II imbalance.

EP 428434 discloses certain alkylureidopyridines as neurokinin and substance P antagonists. WO-99/21835 discloses certain ureidoquinolines as H+-ATPase and bone resorption inhibitors. WO-01/009088 discloses certain substituted heteroarylureas as inhibitors of the CCR-3 receptor. JP-96/061621 discloses certain propionylpiperazines as anticholecystokinin compounds. All of these ureidopyridine derivatives differ in their composition from compounds of the present invention. The present invention comprises N-(2-piperazin-1-yl-ethyl)-N′-pyridin-4-yl urea derivatives which are novel compositions of matter and which are useful as urotensin II receptor antagonists.

### DESCRIPTION OF THE INVENTION

The present invention relates to compounds of the general formula 1. 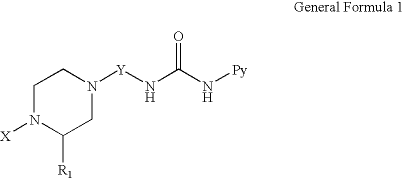
wherein:
Py represents pyridin-4-yl which is disubstituted in positions 2 and 6 or mono-substituted in position 2, whereby the substituent in position 2 is —NR²R³, lower alkyl, aryl-lower alkyl, or (E)-2-aryl-ethen-1-yl, and the substituent in position 6 is hydrogen, lower alkyl or aryl-lower alkyl; unsubstituted quinolin-4-yl; quinolin-4-yl mono-substituted in position 2 with lower alkyl; quinolin-4-yl di-substituted in position 2 with lower alkyl and in position 6, 7, or 8 with halogen, lower alkyl, or aryl-lower alkyl;
X represents aryl; aryl-lower alkyl-; lower alkyl disubstituted with aryl; lower alkyl-SO₂—; aryl-SO₂—; aryl-lower alkyl-SO₂—; lower alkyl-CO—; aryl-CO—; aryl-lower alkyl-CO—; lower alkyl-NR⁶CO—; aryl-NR⁶CO— and aryl-lower alkyl-NR⁶CO—.
Y represents —C(R⁴)(R⁵)(CH₂)— or —(CH₂)—C(R⁴)(R⁵)—.
R¹ represents hydrogen or a methyl group;
R² and R³ represent independently hydrogen; lower alkyl; aryl-lower alkyl; or form together with the nitrogen atom to which they are attached a pyrrolidine, piperidine, or morpholine ring;
R⁴ represents hydrogen; lower alkyl; aryl; aryl-lower alkyl; or forms together with R⁵ a 3-, 4-, 5-, or 6-membered saturated carbocyclic ring including the carbon atom to which R⁴ and R⁵ are attached as ring atoms;
R⁵ represents hydrogen; methyl; or forms together with R⁴ a 3-, 4-, 5-, or 6-membered saturated carbocyclic ring including the carbon atom to which R⁴ and R⁵ are attached as ring atoms;
R⁶ represents hydrogen; lower alkyl; or aryl-lower alkyl;
and optically pure enantiomers or diastereomers, mixtures of enantiomers or diastereomers, diastereomeric racemates, and mixtures of diastereomeric racemates; as well as their pharmaceutically acceptable salts, solvent complexes, and morphological forms.

In the definitions of the general formula 1 the expression ‘aryl’ means a substituted or unsubstituted aromatic carbocyclic or heterocyclic ring system, consisting of a five- or six-membered aromatic ring, or of a fused five-six or six-six aromatic ring system. Preferred aryl groups are for example 2-furyl; 2-thienyl; phenyl; 2-methylphenyl; 2-biphenyl; 2-methoxyphenyl; 2-phenoxyphenyl; 2-chlorophenyl; 2-bromophenyl; 2-i-propylphenyl; 2-fluorophenyl; 2-methylsulfonylphenyl; 2-cyanophenyl; 2-trifluoromethylphenyl; 3-methylphenyl; 3-biphenyl; 3-phenoxyphenyl; 3-methoxyphenyl; 3-chlorophenyl; 3-bromophenyl; 3-fluorophenyl; 3-cyanophenyl; 3-trifluoromethylphenyl; 3-carboxyphenyl; 4-methylphenyl; 4-ethylphenyl; 4-i-propylphenyl; 4-phenyloxyphenyl; 4-methoxyphenyl 4-trifluoromethylphenyl; 4-trifluoromethoxyphenyl; 4-phenoxyphenyl; 4-cyanophenyl; 4-hydroxyphenyl; 4-acetylaminophenyl; 4-methanesulfonylphenyl; 4-n-propylphenyl; 4-isopropylphenyl; 4-tert-butylphenyl; 4-n-pentylphenyl; 4-biphenyl; 4-chlorophenyl; 4-bromophenyl; 4-bromo-2-ethylphenyl; 4-fluorophenyl; 2,4-difluorophenyl; 4-n-butoxyphenyl; 2,6-dimethoxyphenyl; 3,5-bis-trifluoromethylphenyl; 2-pyridyl; 3-pyridyl; 4-pyridyl; 1-naphthyl; 2-naphthyl; 4-(pyrrol-1-yl)phenyl; 4-benzoylphenyl; 5-dimethylaminonaphth-1-yl; 5-chloro-3-methylthiophen-2-yl; 5-chloro-3-methyl-benzo[b]thiophen-2-yl; 3-(phenylsulfonyl)-thiophen-2-yl; 2-(2,2,2-trifluoroacetyl)-1-2,3,4-tetrahydroisoquinolin-7-yl; 4-(3-chloro-2-cyanophenyloxy)phenyl; 2-(5-benzamidomethyl)thiophenyl; 4,5-dichlorothien-2-yl; 5-quinolyl-; 6-quinolyl; 7-quinolyl; 8-quinolyl; (2-acetylamino-4-methyl)thiazol-5-yl; or 1-methylimidazol-4-yl.

In the definitions of the general formula 1 the expression ‘lower alkyl’ means a saturated straight chain, branched chain or cyclic substituent consisting of from one to eight carbons, comprising methyl, ethyl, n-propyl, iso-propyl, n-butyl, iso-butyl, n-pentyl, n-hexyl, n-octyl, cyclopropyl, cyclobutyl, cyclopentyl, cyclohexyl, cyclopropylmethyl and the like. Preferred lower alkyl groups are methyl, ethyl, and n-propyl.

The expression ‘lower alkyl disubstituted with aryl’ means a lower alkyl group as previously defined in which two hydrogen atoms have been replaced by an aryl group as previously defined. Preferred examples of ‘lower alkyl disubstituted with aryl’ groups are diphenylmethyl, 2,2-diphenylethyl and 1-benzyl-2-phenyl-ethyl.

The expression ‘aryl-lower alkyl’ means a lower alkyl group as previously defined in which one hydrogen atom has been replaced by an aryl group as previously defined. Preferred examples of aryl-lower alkyl groups are benzyl, phenethyl and 3-phenylpropyl.

The expression ‘halogen’ encompasses fluoro, chloro, bromo or iodo.

The present invention encompasses pharmaceutically acceptable salts of compounds of the general formula 1. This encompasses either salts with inorganic acids or organic acids like hydrohalogenic acids, e.g. hydrochloric or hydrobromic acid, sulfuric acid, phosphoric acid, nitric acid, citric acid, formic acid, acetic acid, maleic acid, tartaric acid, methylsulfonic acid, p-tolylsulfonic acid and the like or in case the compound of formula 1 is acidic in nature with an inorganic base like an alkali or earth alkali base, e.g. sodium, potassium, or calcium salts, etc. The compounds of general formula 1 can also be present in form of zwitterions.

The present invention encompasses different solvation complexes of compounds of general formula 1. The solvation can be effected in the course of the manufacturing process or can take place separately, e.g. as a consequence of hygroscopic properties of an initially anhydrous compound of general formula 1.

The present invention further encompasses different morphological forms, e.g. crystalline forms of compounds of general formula 1 and their salts and solvation complexes. Particular heteromorphs may exhibit different dissolution properties, stability profiles, and the like, and are all included in the scope of the present invention.

The compounds of the general formula 1 might have one or more asymmetric carbon atoms and may be prepared in form of optically pure enantiomers or diastereomers, mixtures of enantiomers or diastereomers, diastereomeric racemates, and mixtures of diastereomeric racemates. The present invention encompasses all these forms. They are prepared by stereoselective synthesis, or by separation of mixtures in a manner known per se, i.e. by column chromatography, thin layer chromatography, HPLC, crystallization, etc.

Preferred compounds of general formula 1 are the compounds wherein R⁴ and R⁵ represent hydrogen and R¹, X and Py have the meaning given in general formula 1.

Another group of preferred compounds of general formula 1 are the compounds wherein R¹ represents hydrogen and Y, X and Py have the meaning given in general formula 1.

Another group of preferred compounds of general formula 1 are the compounds wherein X represents aryl, aryl-lower alkyl- or lower alkyl disubstituted with aryl-, and R¹, Y and Py have the meaning given in general formula 1.

Another group of preferred compounds of general formula 1 are the compounds wherein X represents aryl-SO₂— or aryl-lower alkyl-SO₂—, and R¹, Y and Py have the meaning given in general formula 1.

Another group of preferred compounds of general formula 1 are the compounds wherein X represents aryl-CO— or aryl-lower alkyl-CO—, and R¹, Y and Py have the meaning given in general formula 1.

Another group of preferred compounds of general formula 1 are the compounds wherein X represents aryl-NR⁶CO— or aryl-lower alkyl-NR⁶CO—, and R¹, R⁶, Y and Py have the meaning given in general formula 1.

Another group of preferred compounds of general formula 1 are the compounds wherein Py represents unsubstituted quinolin-4-yl or quinolin-4-yl mono-substituted in position 2 with lower alkyl, and R¹, X and Y have the meaning given in general formula 1.

Another group of preferred compounds of general formula 1 are the compounds wherein Py represents pyridin-4-yl, substituted in position 2 with R²R³N—, wherein R³ represents aryl-lower alkyl and R² represents lower alkyl, and R¹, X and Y have the meaning given in general formula 1.

Another group of preferred compounds of general formula 1 are the compounds wherein Py represents pyridin-4-yl, substituted in position 2 with R²R³N—, wherein R² represents hydrogen, and R¹, R³, X and Y have the meaning given in general formula 1.

A group of especially preferred compounds of general formula 1 are the compounds wherein R⁴ and R⁵ represent hydrogen, X represents aryl, aryl-lower alkyl- or lower alkyl disubstituted with aryl-, and R¹ and Py have the meaning given in general formula 1.

Another group of especially preferred compounds of general formula 1 are the compounds wherein R¹, R⁴ and R⁵ represent hydrogen, X represents aryl-SO₂— or aryl-lower alkyl-SO₂—, and Py has the meaning given in general formula 1.

Another group of especially preferred compounds of general formula 1 are the compounds wherein R¹, R⁴ and R⁵ represent hydrogen, X represents aryl-CO— or aryl-lower alkyl-CO—, and Py has the meaning given in general formula 1.

Another group of especially preferred compounds of general formula 1 are the compounds wherein R¹, R⁴ and R⁵ represent hydrogen, X represents aryl-NR⁶CO— or aryl-lower alkyl-NR⁶CO—, and R⁶ and Py have the meaning given in general formula 1.

Another group of especially preferred compounds of general formula 1 are the compounds wherein R¹, R⁴ and R⁵ represent hydrogen, Py represents unsubstituted quinolin-4-yl or quinolin-4-yl mono-substituted in position 2 with lower alkyl, and X has the meaning given in general formula 1.

Another group of especially preferred compounds of general formula 1 are the compounds wherein R¹, R⁴ and R⁵ represent hydrogen, Py represents pyridin-4-yl, substituted in position 2 with R²R³N—, wherein R³ represents aryl-lower alkyl and R² represents lower alkyl, and X has the meaning given in general formula 1.

Another group of especially preferred compounds of general formula 1 are the compounds wherein R¹, R⁴ and R⁵ represent hydrogen, Py represents pyridin-4-yl, substituted in position 2 with R²R³N—, wherein R² represents hydrogen, and R³ and X have the meaning given in general formula 1.

A group of most preferred compounds of general formula 1 are the compounds wherein R¹, R⁴ and R⁵ represent hydrogen, X represents aryl, aryl-lower alkyl- or lower alkyl disubstituted with aryl-, and Py represents unsubstituted quinolin-4-yl or quinolin-4-yl mono-substituted in position 2 with lower alkyl.

Another group of most preferred compounds of general formula 1 are the compounds wherein R¹, R⁴ and R⁵ represent hydrogen, X represents aryl-SO₂— or aryl-lower alkyl-SO₂—, and Py represents unsubstituted quinolin-4-yl or quinolin-4-yl mono-substituted in position 2 with lower alkyl.

Another group of most preferred compounds of general formula 1 are the compounds wherein R¹, R⁴ and R⁵ represent hydrogen, X represents aryl-CO— or aryl-lower alkyl-CO—, and Py represents unsubstituted quinolin-4-yl or quinolin-4-yl mono-substituted in position 2 with lower alkyl.

Another group of most preferred compounds of general formula 1 are the compounds wherein R¹, R⁴ and R⁵ represent hydrogen, X represents aryl-NR⁶CO— or aryl-lower alkyl-NR⁶CO—, Py represents unsubstituted quinolin-4-yl or quinolin-4-yl mono-substituted in position 2 with lower alkyl, and R⁶ has the meaning given in general formula 1.

Examples of preferred compounds of general formula 1 are selected from the list consisting of:

| Example | |
|---|---|
| Number | |
| 1. | 1-[2-(4-Benzenesulfonyl-piperazin-1-yl)-ethyl]-3-(2-methyl- |
| | quinolin-4-yl)-urea |
| 2. | 1-(2-Methyl-quinolin-4-yl)-3-{2-[4-(thiophene-2-sulfonyl)-piperazin- |
| | 1-yl]-ethyl}-urea |
| 3. | 1-(2-Methyl-quinolin-4-yl)-3-{2-[4-(toluene-3-sulfonyl)-piperazin-1- |
| | yl]-ethyl}-urea |
| 4. | 1-(2-Methyl-quinolin-4-yl)-3-{2-[4-(toluene-2-sulfonyl)-piperazin-1- |
| | yl]-ethyl}-urea |
| 5. | 1-{2-[4-(2-Fluoro-benzenesulfonyl)-piperazin-1-yl]-ethyl}-3-(2- |
| | methyl-quinolin-4-yl)-urea |
| 6. | 1-{2-[4-(3-Fluoro-benzenesulfonyl)-piperazin-1-yl]-ethyl}-3-(2- |
| | methyl-quinolin-4-yl)-urea |
| 7. | 1-{2-[4-(4-Fluoro-benzenesulfonyl)-piperazin-1-yl]-ethyl}-3-(2- |
| | methyl-quinolin-4-yl)-urea |
| 8. | 1-{2-[4-(3-Cyano-benzenesulfonyl)-piperazin-1-yl]-ethyl}-3-(2- |
| | methyl-quinolin-4-yl)-urea |
| 9. | 1-{2-[4-(4-Cyano-benzenesulfonyl)-piperazin-1-yl]-ethyl}-3-(2- |
| | methyl-quinolin-4-yl)-urea |
| 10. | 1-{2-[4-(3-Methoxy-benzenesulfonyl)-piperazin-1-yl]-ethyl}-3-(2- |
| | methyl-quinolin-4-yl)-urea |
| 11. | 1-{2-[4-(3-Chloro-benzenesulfonyl)-piperazin-1-yl]-ethyl}-3-(2- |
| | methyl-quinolin-4-yl)-urea |
| 12. | 1-{2-[4-(4-Chloro-benzenesulfonyl)-piperazin-1-yl]-ethyl}-3-(2- |
| | methyl-quinolin-4-yl)-urea |
| 13. | 1-{2-[4-(2-Chloro-benzenesulfonyl)-piperazin-1-yl]-ethyl}-3-(2- |
| | methyl-quinolin-4-yl)-urea |
| 14. | 3-(4-{2-[3-(2-Methyl-quinolin-4-yl)-ureido]-ethyl}-piperazine-1- |
| | sulfonyl)-benzoic acid |
| 15. | 1-(2-Methyl-quinolin-4-yl)-3-{2-[4-(naphthalene-2-sulfonyl)- |
| | piperazin-1-yl]-ethyl}-urea |
| 16. | 1-(2-Methyl-quinolin-4-yl)-3-{2-[4-(naphthalene-1-sulfonyl)- |
| | piperazin-1-yl]-ethyl}-urea |
| 17. | 1-(2-Methyl-quinolin-4-yl)-3-{2-[4-(quinoline-8-sulfonyl)-piperazin- |
| | 1-yl]-ethyl}-urea |
| 18. | 1-(2-Methyl-quinolin-4-yl)-3-{2-[4-(4-trifluoromethyl- |
| | benzenesulfonyl)-piperazin-1-yl]-ethyl}-urea |
| 19. | 1-(2-Methyl-quinolin-4-yl)-3-{2-[4-(2-trifluoromethyl- |
| | benzenesulfonyl)-piperazin-1-yl]-ethyl}-urea |
| 20. | 1-(2-Methyl-quinolin-4-yl)-3-{2-[4-(3-trifluoromethyl- |
| | benzenesulfonyl)-piperazin-1-yl]-ethyl}-urea |
| 21. | 1-{2-[4-(3,4-Dichloro-benzenesulfonyl)-piperazin-1-yl]-ethyl}-3-(2-methyl- |
| | quinolin-4-yl)-urea |
| 22. | 1-{2-[4-(4-Butoxy-benzenesulfonyl)-piperazin-1-yl]-ethyl}-3-(2- |
| | methyl-quinolin-4-yl)-urea |
| 23. | 1-{2-[4-(4,5-Dichloro-thiophene-2-sulfonyl)-piperazin-1-yl]-ethyl}-3-(2-methyl-quinolin- |
| | 4-yl)-urea |
| 24. | 1-(2-{4-[4-(3-Chloro-2-cyano-phenoxy)-benzenesulfonyl]- |
| | piperazin-1-yl}-ethyl)-3-(2-methyl-quinolin-4-yl)-urea |
| 25. | 1-{2-[4-(2-Methanesulfonyl-benzenesulfonyl)-piperazin-1-yl]- |
| | ethyl}-3-(2-methyl-quinolin-4-yl)-urea |
| 26. | N-[4-Methyl-5-(4-{2-[3-(2-methyl-quinolin-4-yl)-ureido]-ethyl}- |
| | piperazine-1-sulfonyl)-thiazol-2-yl]-acetamide |
| 27. | 1-{2-[4-(3-Bromo-benzenesulfonyl)-piperazin-1-yl]-ethyl}-3-(2- |
| | methyl-quinolin-4-yl)-urea |
| 28. | 1-{2-[4-(4-Bromo-benzenesulfonyl)-piperazin-1-yl]-ethyl}-3-(2- |
| | methyl-quinolin-4-yl)-urea |
| 29. | 1-(2-Methyl-quinolin-4-yl)-3-{2-[4-(2-trifluoromethoxy- |
| | benzenesulfonyl)-piperazin-1-yl]-ethyl}-urea |
| 30. | 1-(2-Methyl-quinolin-4-yl)-3-{2-[4-(4-trifluoromethoxy- |
| | benzenesulfonyl)-piperazin-1-yl]-ethyl}-urea |
| 31. | 1-{2-[4-(5-Dimethylamino-naphthalene-1-sulfonyl)-piperazin-1-yl]- |
| | ethyl}-3-(2-methyl-quinolin-4-yl)-urea |
| 32. | 1-{2-[4-(5-Chloro-3-methyl-benzo[b]thiophene-2-sulfonyl)- |
| | piperazin-1-yl]-ethyl}-3-(2-methyl-quinolin-4-yl)-urea |
| 33. | 1-{2-[4-(4-Bromo-2-ethyl-benzenesulfonyl)-piperazin-1-yl]-ethyl}- |
| | 3-(2-methyl-quinolin-4-yl)-urea |
| 34. | 1-{2-[4-(3,5-Bis-trifluoromethyl-benzenesulfonyl)-piperazin-1-yl]- |
| | ethyl}-3-(2-methyl-quinolin-4-yl)-urea |
| 35. | N-[5-(4-{2-[3-(2-Methyl-quinolin-4-yl)-ureido]-ethyl}-piperazine-1- |
| | sulfonyl)-thiophen-2-ylmethyl]-benzamide |
| 36. | 1-{2-[4-(4-Benzenesulfonyl-thiophene-2-sulfonyl)-piperazin-1-yl]- |
| | ethyl}-3-(2-methyl-quinolin-4-yl)-urea |
| 37. | 1-(2-Methyl-quinolin-4-yl)-3-(2-{4-[2-(2,2,2-trifluoro-acetyl)-1,2,3,4- |
| | tetrahydro-isoquinoline-7-sulfonyl]-piperazin-1-yl}-ethyl)-urea |
| 38. | 1-(2-Methyl-quinolin-4-yl)-3-[2-(4-phenylmethanesulfonyl- |
| | piperazin-1-yl)-ethyl]-urea |
| 39. | 1-(2-Methyl-quinolin-4-yl)-3-{2-[4-(octane-1-sulfonyl)-piperazin-1- |
| | yl]-ethyl}-urea |
| 40. | 1-{2-[4-(7,7-Dimethyl-2-oxo-bicyclo[2.2.1]hept-1- |
| | ylmethanesulfonyl)-piperazin-1-yl]-ethyl}-3-(2-methyl-quinolin-4- |
| | yl)-urea |
| 41. | 4-{2-[3-(2-Methyl-quinolin-4-yl)-ureido]-ethyl}-piperazine-1- |
| | carboxylic acid biphenyl-2-ylamide |
| 42. | 4-{2-[3-(2-Methyl-quinolin-4-yl)-ureido]-ethyl}-piperazine-1- |
| | carboxylic acid (4-phenoxy-phenyl)-amide |
| 43. | 4-{2-[3-(2-Methyl-quinolin-4-yl)-ureido]-ethyl}-piperazine-1- |
| | carboxylic acid cyclohexylamide |
| 44. | 4-{2-[3-(2-Methyl-quinolin-4-yl)-ureido]-ethyl}-piperazine-1- |
| | carboxylic acid m-tolylamide |
| 45. | 4-{2-[3-(2-Methyl-quinolin-4-yl)-ureido]-ethyl}-piperazine-1- |
| | carboxylic acid (4-methoxy-phenyl)-amide |
| 46. | 4-{2-[3-(2-Methyl-quinolin-4-yl)-ureido]-ethyl}-piperazine-1- |
| | carboxylic acid (2-methoxy-phenyl)-amide |
| 47. | 4-{2-[3-(2-Methyl-quinolin-4-yl)-ureido]-ethyl}-piperazine-1- |
| | carboxylic acid o-tolylamide |
| 48. | 4-{2-[3-(2-Methyl-quinolin-4-yl)-ureido]-ethyl}-piperazine-1- |
| | carboxylic acid (3-chloro-phenyl)-amide |
| 49. | 4-{2-[3-(2-Methyl-quinolin-4-yl)-ureido]-ethyl}-piperazine-1- |
| | carboxylic acid naphthalen-2-ylamide |
| 50. | 4-{2-[3-(2-Methyl-quinolin-4-yl)-ureido]-ethyl}-piperazine-1- |
| | carboxylic acid (2,4-difluoro-phenyl)-amide |
| 51. | 4-{2-[3-(2-Methyl-quinolin-4-yl)-ureido]-ethyl}-piperazine-1- |
| | carboxylic acid butylamide |
| 52. | 4-{2-[3-(2-Methyl-quinolin-4-yl)-ureido]-ethyl}-piperazine-1- |
| | carboxylic acid benzylamide |
| 53. | 4-{2-[3-(2-Methyl-quinolin-4-yl)-ureido]-ethyl}-piperazine-1- |
| | carboxylic acid (2-fluoro-phenyl)-amide |
| 54. | 4-{2-[3-(2-Methyl-quinolin-4-yl)-ureido]-ethyl}-piperazine-1- |
| | carboxylic acid (4-fluoro-phenyl)-amide |
| 55. | 4-{2-[3-(2-Methyl-quinolin-4-yl)-ureido]-ethyl}-piperazine-1- |
| | carboxylic acid (3-fluoro-phenyl)-amide |
| 56. | 4-{2-[3-(2-Methyl-quinolin-4-yl)-ureido]-ethyl}-piperazine-1- |
| | carboxylic acid phenethyl-amide |
| 57. | 4-{2-[3-(2-Methyl-quinolin-4-yl)-ureido]-ethyl}-piperazine-1- |
| | carboxylic acid (3-methoxy-phenyl)-amide |
| 58. | 4-{2-[3-(2-Methyl-quinolin-4-yl)-ureido]-ethyl}-piperazine-1- |
| | carboxylic acid 4-fluoro-benzylamide |
| 59. | 4-{2-[3-(2-Methyl-quinolin-4-yl)-ureido]-ethyl}-piperazine-1- |
| | carboxylic acid (2-isopropyl-phenyl)-amide |
| 60. | 4-{2-[3-(2-Methyl-quinolin-4-yl)-ureido]-ethyl}-piperazine-1- |
| | carboxylic acid (4-bromo-phenyl)-amide |
| 61. | 4-{2-[3-(2-Methyl-quinolin-4-yl)-ureido]-ethyl}-piperazine-1- |
| | carboxylic acid (2-phenoxy-phenyl)-amide |
| 62. | 4-{2-[3-(2-Methyl-quinolin-4-yl)-ureido]-ethyl}-piperazine-1- |
| | carboxylic acid (4-chloro-phenyl)-amide |
| 63. | 1-(2-{4-[2-(4-Chloro-phenyl)-acetyl]-piperazin-1-yl}-ethyl)-3-(2- |
| | methyl-quinolin-4-yl)-urea |
| 64. | 1-(2-{4-[2-(4-Methoxy-phenyl)-acetyl]-piperazin-1-yl}-ethyl)-3-(2- |
| | methyl-quinolin-4-yl)-urea |
| 65. | 1-(2-Methyl-quinolin-4-yl)-3-{2-[4-(naphthalene-2-carbonyl)- |
| | piperazin-1-yl]-ethyl}-urea |
| 66. | 1-(2-{4-[2-(4-Isopropyl-phenyl)-acetyl]-piperazin-1-yl}-ethyl)-3-(2- |
| | methyl-quinolin-4-yl)-urea |
| 67. | 1-(2-Methyl-quinolin-4-yl)-3-{2-[4-(2-naphthalen-1-yl-acetyl)- |
| | piperazin-1-yl]-ethyl}-urea |
| 68. | 1-[2-(4-Benzoyl-piperazin-1-yl)-ethyl]-3-(2-methyl-quinolin-4-yl)- |
| | urea |
| 69. | 1-(2-{4-[3-(4-Fluoro-phenyl)-propionyl]-piperazin-1-yl}-ethyl)-3-(2- |
| | methyl-quinolin-4-yl)-urea |
| 70. | 1-(2-Methyl-quinolin-4-yl)-3-{2-[4-(3-phenyl-propionyl)-piperazin- |
| | 1-yl]-ethyl}-urea |
| 71. | 1-(2-{4-[2-(4-Fluoro-phenyl)-acetyl]-piperazin-1-yl}-ethyl)-3-(2- |
| | methyl-quinolin-4-yl)-urea |
| 72. | 1-(2-Methyl-quinolin-4-yl)-3-{2-[4-(3-p-tolyl-propionyl)-piperazin-1- |
| | yl]-ethyl}-urea |
| 73. | 1-(2-{4-[3-(2-Methoxy-phenyl)-propionyl]-piperazin-1-yl}-ethyl)-3- |
| | (2-methyl-quinolin-4-yl)-urea |
| 74. | 1-(2-{4-[3-(4-Methoxy-phenyl)-propionyl]-piperazin-1-yl}-ethyl)-3- |
| | (2-methyl-quinolin-4-yl)-urea |
| 75. | 1-(2-{4-[3-(3-Methoxy-phenyl)-propionyl]-piperazin-1-yl}-ethyl)-3- |
| | (2-methyl-quinolin-4-yl)-urea |
| 76. | 1-(2-Methyl-quinolin-4-yl)-3-[2-(4-phenylacetyl-piperazin-1-yl)- |
| | ethyl]-urea |
| 77. | 1-(2-Methyl-quinolin-4-yl)-3-{2-[4-(2-m-tolyl-acetyl)-piperazin-1-yl]- |
| | ethyl}-urea |
| 78. | 1-(2-Methyl-quinolin-4-yl)-3-{2-[4-(2-p-tolyl-acetyl)-piperazin-1-yl]- |
| | ethyl}-urea |
| 79. | 1-{2-[4-(3-Chloro-benzoyl)-piperazin-1-yl]-ethyl}-3-(2-methyl- |
| | quinolin-4-yl)-urea |
| 80. | 1-(2-Methyl-quinolin-4-yl)-3-{2-[4-(quinoline-6-carbonyl)-piperazin- |
| | 1-yl]-ethyl}-urea |
| 81. | 1-{2-[4-(4-tert-Butyl-benzoyl)-piperazin-1-yl]-ethyl}-3-(2-methyl- |
| | quinolin-4-yl)-urea |
| 82. | 1-(2-{4-[2-(4-Dimethylamino-phenyl)-acetyl]-piperazin-1-yl}-ethyl)- |
| | 3-(2-methyl-quinolin-4-yl)-urea |
| 83. | 1-{2-[4-(2,4-Dimethoxy-benzoyl)-piperazin-1-yl]-ethyl}-3-(2- |
| | methyl-quinolin-4-yl)-urea |
| 84. | 1-(2-Methyl-quinolin-4-yl)-3-{2-[4-(4-pyrrol-1-yl-benzoyl)- |
| | piperazin-1-yl]-ethyl}-urea |
| 85. | 1-(2-{4-[2-(4-Bromo-phenyl)-acetyl]-piperazin-1-yl}-ethyl)-3-(2- |
| | methyl-quinolin-4-yl)-urea |
| 86. | 1-{2-[4-(4-Benzoyl-benzoyl)-piperazin-1-yl]-ethyl}-3-(2-methyl- |
| | quinolin-4-yl)-urea |
| 87. | 1-[2-(4-Benzyl-piperazin-1-yl)-ethyl]-3-quinolin-4-yl-urea |
| 88. | 1-[2-(4-Benzyl-piperazin-1-yl)-ethyl]-3-pyridin-4-yl-urea |
| 89. | 1-{2-[4-(4-Methoxy-phenyl)-3-methyl-piperazin-1-yl]-ethyl}-3- |
| | quinolin-4-yl-urea |
| 90. | 1-{2-[4-(2-Methoxy-phenyl)-piperazin-1-yl]-ethyl}-3-quinolin-4-yl- |
| | urea |
| 91. | 1-{2-[4-(3-Methoxy-phenyl)-piperazin-1-yl]-ethyl}-3-quinolin-4-yl- |
| | urea |
| 92. | 1-[2-(4-Phenyl-piperazin-1-yl)-ethyl]-3-quinolin-4-yl-urea |
| 93. | 1-[2-(3-Methyl-4-p-tolyl-piperazin-1-yl)-ethyl]-3-quinolin-4-yl-urea |
| 94. | 1-Quinolin-4-yl-3-[2-(4-m-tolyl-piperazin-1-yl)-ethyl]-urea |
| 95. | 1-{2-[4-(4-Fluoro-phenyl)-piperazin-1-yl]-ethyl}-3-quinolin-4-yl- |
| | urea |
| 96. | 1-[2-(4-Benzo[1,3]dioxol-5-ylmethyl-piperazin-1-yl)-ethyl]-3- |
| | quinolin-4-yl-urea |
| 97. | 1-{2-[4-(2-Fluoro-phenyl)-piperazin-1-yl]-ethyl}-3-quinolin-4-yl- |
| | urea |
| 98. | 1-Quinolin-4-yl-3-{2-[4-(3-trifluoromethyl-phenyl)-piperazin-1-yl]- |
| | ethyl}-urea |
| 99. | 1-Quinolin-4-yl-3-[2-(4-o-tolyl-piperazin-1-yl)-ethyl]-urea |
| 100. | 1-{2-[4-(3-Chloro-phenyl)-piperazin-1-yl]-ethyl}-3-quinolin-4-yl- |
| | urea |
| 101. | 1-[2-(4-Benzhydryl-piperazin-1-yl)-ethyl]-3-quinolin-4-yl-urea |

Because of their ability to inhibit the actions of urotensin II, the described compounds can be used for treatment of diseases which are associated with an increase in vasoconstriction, proliferation or other disease states associated with the actions of urotensin II. Examples of such diseases are hypertension, atherosclerosis, angina or myocardial ischemia, congestive heart failure, cardiac insufficiency, cardiac arrhythmias, renal ischemia, chronic kidney disease, renal failure, stroke, cerebral vasospasm, cerebral ischemia, dementia, migraine, subarachnoidal hemorrhage, diabetes, diabetic arteriopathy, diabetic nephropathy, connective tissue diseases, cirrhosis, asthma, chronic obstructive pulmonary disease, high-altitude pulmonary edema, Raynaud's syndrome, portal hypertension, thyroid dysfunction, pulmonary edema, pulmonary hypertension, or pulmonary fibrosis. They can also be used for prevention of restenosis after balloon or stent angioplasty, for the treatment of cancer, prostatic hypertrophy, erectile dysfunction, hearing loss, amaurosis, chronic bronchitis, asthma, gram negative septicemia, shock, sickle cell anemia, sickle cell acute chest syndrome, glomerulonephritis, renal colic, glaucoma, therapy and prophylaxis of diabetic complications, complications of vascular or cardiac surgery or after organ transplantation, complications of cyclosporin treatment, pain, addictions, schizophrenia, Alzheimer's disease, anxiety, obsessive-compulsive behavior, epileptic seizures, stress, depression, dementias, neuromuscular disorders, neurodegenerative diseases, as well as other diseases related to a dysregulation of urotensin II or urotensin II receptors.

These compositions may be administered in enteral or oral form e.g. as tablets, dragees, gelatine capsules, emulsions, solutions or suspensions, in nasal form like sprays and aerosols, or rectally in form of suppositories. These compounds may also be administered in intramuscular, parenteral or intravenous form, e.g. in form of injectable solutions.

These pharmaceutical compositions may contain the compounds of formula 1 as well as their pharmaceutically acceptable salts in combination with inorganic and/or organic excipients, which are usual in the pharmaceutical industry, like lactose, maize or derivatives thereof, talcum, stearic acid or salts of these materials.

For gelatine capsules vegetable oils, waxes, fats, liquid or half-liquid polyols etc. may be used. For the preparation of solutions and sirups e.g. water, polyols, saccharose, glucose etc. are used. Injectables are prepared by using e.g. water, polyols, alcohols, glycerin, vegetable oils, lecithin, liposomes etc. Suppositories are prepared by using natural or hydrogenated oils, waxes, fatty acids (fats), liquid or half-liquid polyols etc.

The compositions may contain in addition preservatives, stabilisation improving substances, viscosity improving or regulating substances, solubility improving substances, sweeteners, dyes, taste improving compounds, salts to change the osmotic pressure, buffer, anti-oxidants etc.

The compounds of general formula 1 may also be used in combination with one or more other therapeutically useful substances e.g. α- and β-blockers like phentolamine, phenoxybenzamine, atenolol, propranolol, timolol, metoprolol, carteolol, carvedilol, etc.; with vasodilators like hydralazine, minoxidil, diazoxide, flosequinan, etc.; with calcium-antagonists like diltiazem, nicardipine, nimodipine, verapamil, nifedipine, etc.; with angiotensin converting enzyme-inhibitors like cilazapril, captopril, enalapril, lisinopril etc.; with potassium channel activators like pinacidil, chromakalim, etc.; with angiotensin receptor antagonists like losartan, valsartan, candesartan, irbesartan, eprosartan, telmisartan, and tasosartan, etc.; with diuretics like hydrochlorothiazide, chlorothiazide, acetolamide, bumetanide, furosemide, metolazone, chlortalidone, etc.; with sympatholytics like methyldopa, clonidine, guanabenz, reserpine, etc.; with endothelin receptor antagonists like bosentan, tezosentan, darusentan, atrasentan, enrasentan, or sitaxsentan, etc.; with anti-hyperlipidemic agents like lovastatin, pravistatin, fluvastatin, atorvastatin, cerivastatin, simvastatin, etc.; and other therapeutics which serve to treat high blood pressure, vascular disease or other disorders listed above.

The dosage may vary within wide limits but should be adapted to the specific situation. In general the dosage given daily in oral form should be between about 1 mg and about 3 g, preferably between about 3 mg and about 1 g, especially preferred between 5 mg and 300 mg, per adult with a body weight of about 70 kg. The dosage should be administered preferably in 1 to 3 doses of equal weight per day. As usual children should receive lower doses which are adapted to body weight and age.

General preparation of Compounds of the Invention

Compounds of the general formula 1 can be prepared using methods generally known in the art, according to the general sequence of reactions outlined below. For simplicity and clarity reasons sometimes only a few of the possible synthetic routes that lead to compounds of general formula 1 are described.

For the synthesis of compounds of general formula 1 general synthetic routes illustrated in Schemes A through E can be employed. The generic groups X, Y, Py, R¹, R², R³, R⁴, R⁵ and R⁶ employed in Schemes A through E have the definitions given in general formula 1 above. Other abbreviations used are defined in the Experimental Section. Some instances of the generic groups X might be incompatible with the assembly illustrated in Schemes A through E and so will require the use of protecting groups. The use of protecting groups is well known in the art (see for example “Protective Groups in Organic Synthesis”, T. W. Greene, P. G. M. Wuts, Wiley-Interscience, 1999). For the purposes of this discussion, it will be assumed that such protecting groups as are necessary are in place.

Preparation of compounds of general formula 1 wherein Y is —(CH₂)—C(R⁴)(R⁵)—. Compounds of general formula 1 wherein Y is —(CH₂)—C(R⁴)(R⁵)— are prepared according to Scheme A. Compounds of general formula 1 wherein Y is —C(R⁴)(R⁵)—(CH₂)— are prepared according to Scheme B.

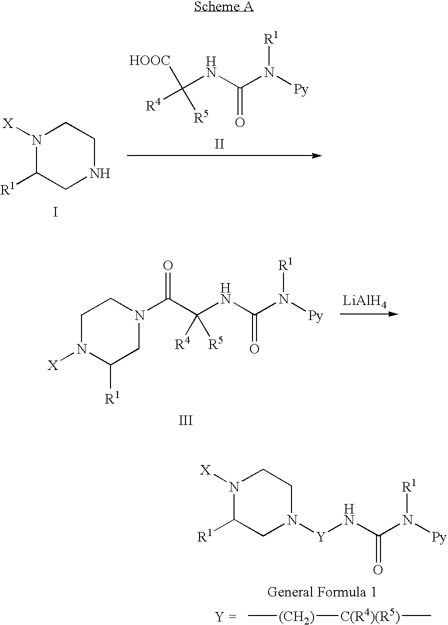

4-Substituted-piperazines of general structure I in Scheme A are either commercially available in racemic or optically active form or are prepared in racemic or optically active form by methods well known in the art. Ureido acetic-derivatives of general structure II in Scheme A are prepared according to Scheme F below. N-Acylation of piperazines of general structure I with ureido acetic acid derivatives of general structure II is accomplished in a polar solvent such as DMF in the presence of a small stoichiometric excess of a coupling reagent such as a carbodiimide to provide amides of general structure III. Selective reduction of the amide carbonyl group with a reagent such as LiAlH₄ in a polar solvent such as THF provides the target compounds of general formula 1 wherein Y is —(CH₂)—C(R⁴)(R⁵)—.

Preparation of compounds of general formula 1. These compounds are alternatively prepared according to Scheme B.

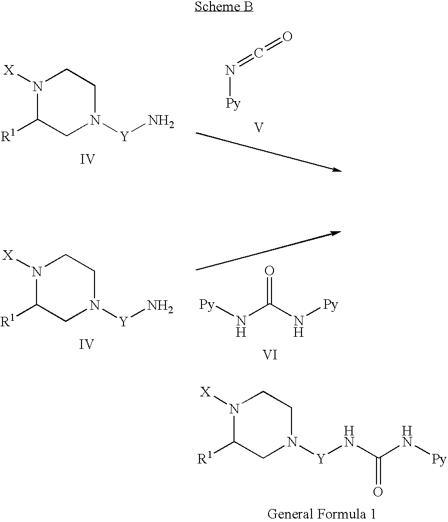

Amines of general structure IV are reacted with isocyanates of general structure V to provide the final compounds of general formula 1. Alternatively, amines of general structure IV are reacted with ureas of general structure VI to provide the final compounds of general formula 1. The preparation of isocyanates of general structure V and of ureas of general structure VI is described in Scheme E below. The preparation of amines of general structure IV is described in Scheme G below.

Compounds of general formula I wherein R⁴ and R⁵ are H. These compounds are alternatively prepared according to the method illustrated in Scheme C.

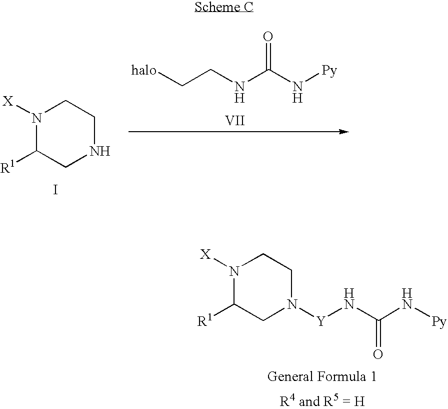

4-Substituted-piperazines of general structure I in Scheme C are either commercially available in racemic or optically active form or are prepared in racemic or optically active form by methods well known in the art. Haloalkyl ureas of general structure VII in Scheme C are prepared according to Scheme E below. N-Alkylation of piperazines of general structure I with haloalkyl ureas of general structure VII is accomplished in a polar solvent such as tetrahydrofuran in the presence of a sub-stoichiometric amount of an iodide salt such as NaI and a small stoichiometric excess of acid scavenger such as NaHCO₃, to provide the target compounds of general formula 1.

Compounds of general formula 1 wherein X represents lower alkyl-SO₂—: aryl-SO₂—; aryl-lower alkyl-SO₂—: lower alkyl-CO—: aryl-CO—: aryl-lower alkyl-CO—: lower alkyl-NR⁶CO—: aryl-NR⁶CO—: aryl-lower alkyl-NR⁶CO—; and R¹, R⁴, R⁵ and R⁶ represent H. These compounds are alternatively prepared according to the method illustrated in Scheme D.

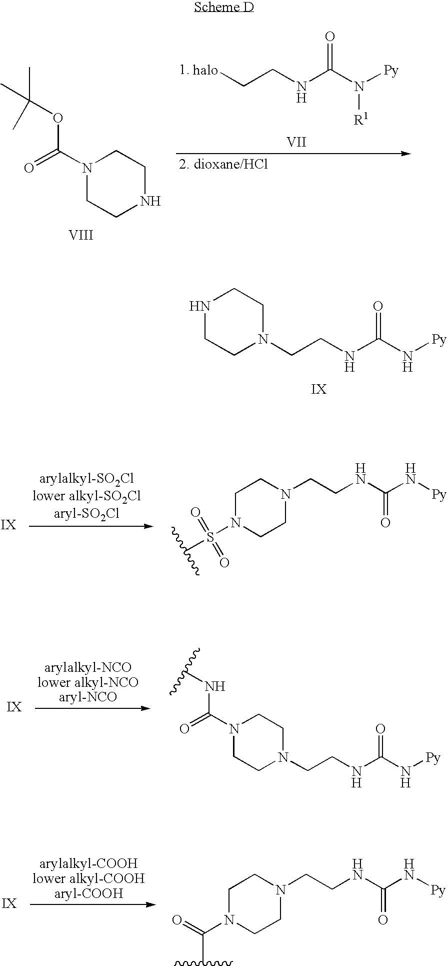

Carbamates of general structure VII are either commercially available or readily prepared by methods well known in the art. Haloalkyl ureas of general structure VII are prepared according to Scheme E below. Carbamates of general structure VIII are reacted with haloalkyl ureas of general structure VII in a polar solvent such as tetrahydrofuran in the presence of a substoichiometric amount of an iodide salt such as NaI and a small stoichiometric excess of an acid scavenger such as NaHCO₃, followed by removal of the carbamate group under acidic conditions, such as reaction with TFA in CH₂Cl₂. The resulting compounds of general structure IX are converted to final compounds of general formula 1 wherein X represents lower alkyl-SO₂—; aryl-SO₂—; aryl-lower alkyl-SO₂—; lower alkyl-CO—; aryl-CO—; aryl-lower alkyl-CO—; lower alkyl-NR⁶CO—; aryl-NR⁶CO—; aryl-lower alkyl-NR⁶CO—; and R¹, R⁴ and R⁵ represent H, by reaction with commercially available or well known sulfonylchlorides, isocyanates, or activated acid derivatives.

Synthetic intermediates used in Schemes A, B, C, and D. Synthetic intermediates containing the group Py, as defined in the general formula 1 above, are obtained by the methods illustrated in Scheme E and F.

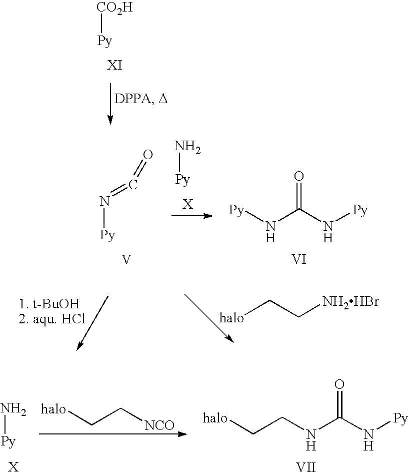

Carboxylic acids of general structure XI are commercially available or are prepared by well known methods. Reaction with diphenylphosphorylazide provides the acyl azide, which undergoes Curtius rearrangement to provide the isocyanates of general structure V, which are used in situ. Reaction of isocyanates of general formula V with amines of general formula X provides ureas of general formula VI. Isocyanates of general structure V, reacted with haloethylamine hydrochloride in the presence of an acid scavenger such as DIPEA, provide ureas of general structure VII. Isocyanates of general structure V are reacted with tert-butanol to provide the corresponding carbamoyl ester, which is hydrolyzed with aqueous acid such as HCl, to provide amines of general structure X. Reaction of amines of general structure X with commercially available chloroethylisocyanate in a polar aprotic solvent such as tetrahydrofuran provides the ureas of general structure VII.

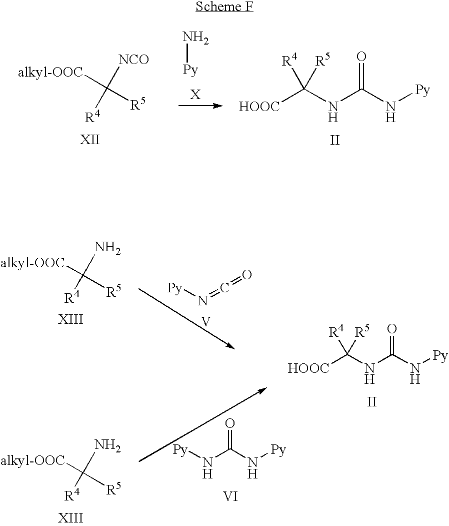

Reaction of amines of general structure X with isocyanates of general structure V provide symmetrical ureas of general structure VI. Reaction of amines of general structure X with commercially available 2-isocyanato-carboxylic acid esters of general formula XII in a polar aprotic solvent such as tetrahydrofuran, followed by hydrolysis of the ester in aqueous acid such as HCl, provides carboxylic acids of general structure II. Alternatively, isocyanates of general structure V and ureas of general structure VI react with amino acid esters of general structure XIII to provide, after hydrolysis of the ester in aqueous acid such as HCl, carboxylic acids of general structure II.

Synthetic intermediates of general structure IV are obtained by the methods illustrated in Scheme G.

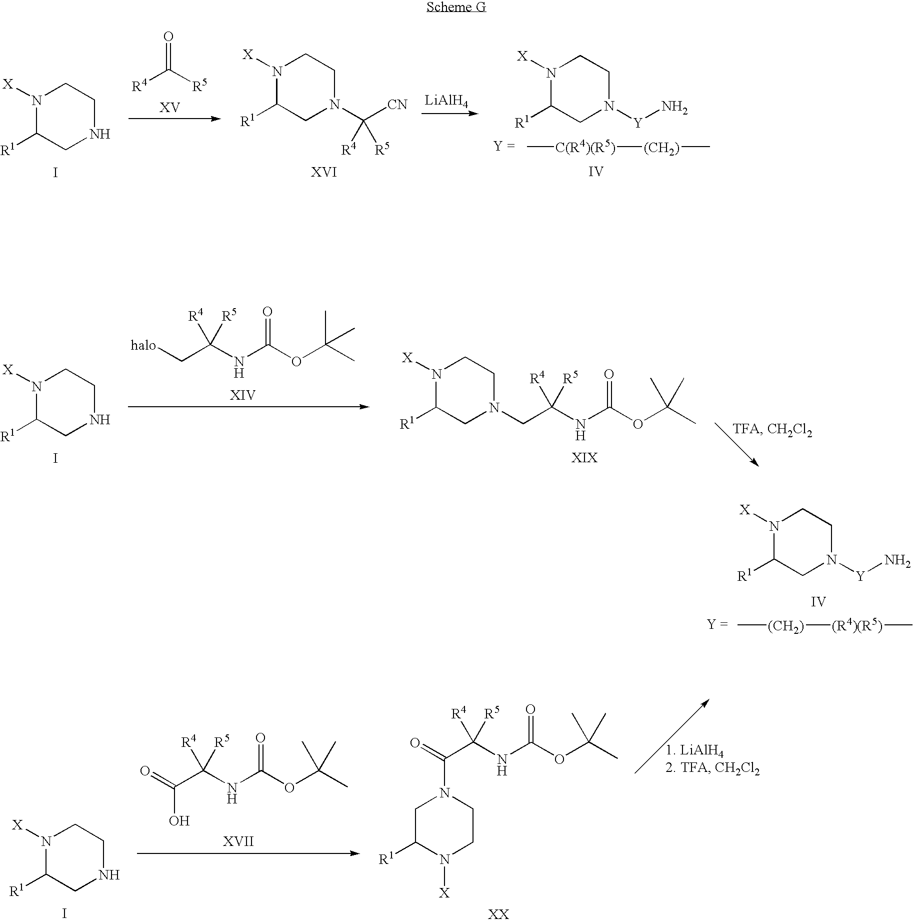

4-Substituted-piperazines of general structure I in Scheme A are either commercially available in racemic or optically active form or are prepared in racemic or optically active form by methods well known in the art. Ketones and aldehydes of general formula XV are commercially available or are prepared by methods well-known in the art. Reaction of ketones and aldehydes of general formula XV with 4-substituted-piperazines of general structure I in presence of a cyanide ion donor such as acetone cyanohydrine provides piperazine derivatives of general structure XVI. Reduction of the cyano group with a reducing reagent such as LiAlH₄ in a polar aprotic solvent such as THF provides the intermediate primary amines of general structure IV, wherein Y is —C(R⁴)(R⁵)—(CH₂)—. Haloalkyl carbamates of general structure XIV in Scheme G are commercially available or are prepared by methods well-known in the art. N-Alkylation of piperazines of general structure I with haloalkyl carbamates of general structure XIV is accomplished in a polar solvent such as THF in the presence of a small stoichiometric excess of acid scavenger such as DIPEA to provide compounds of general structure XIX. Cleavage of the resulting carbamate with methods well known in the art, for example with TFA in a solvent such as CH₂Cl₂, provides the intermediate primary amine derivatives of general structure IV wherein Y is —(CH₂)—C(R⁴)(R⁵)—. Protected amino acids of general structure XVII are commercially available or are prepared by methods well-known in the art. N-Acylation of piperazines of general structure IV with compounds of general structure XVII is accomplished under well-known conditions, for example in a polar solvent such as DMF in the presence of a small stoichiometric excess of a coupling agent such as a carbodiimide, to provide compounds of general structure XX. Reduction with a reagent such as LiAlH₄ and deprotection provides intermediate primary amines of general structure IV wherein Y is —(CH₂)—C(R⁴)(R⁵)—.

The foregoing general description of the invention will now be further illustrated with a number of non-limiting examples.

### EXAMPLES OF THE INVENTION

### List Of Abbreviations

BSA bovine serum albumin

CDI N,N-carbonyldiimidazole

DIPEA diisopropylethylamine

DMF dimethylformamide

DMSO dimethylsulfoxide

DPPA diphenylphosphoryl azide

EDC N-(3-dimethylaminopropyl)-N′-ethylcarbodiimide hydrochloride

EDTA ethylenediamine tetra-acetic acid

ESI electrospray ionization

EtOAc ethyl acetate

Hex hexane

HOBt 1-hydroxybenzotriazole

AcOH acetic acid

HPLC high performance liquid chromatography

LC-MS liquid chromatography-mass spectroscopy

LDA lithium diisopropylamide

MeOH methanol

min minutes

MHz megahertz

MS mass spectroscopy

NMR nuclear magnetic resonance

ppm part per million

PBS phosphate-buffered saline

sat. saturated

TBTU 2-(1H-benzotriazole-1-yl)-1,1,3,3-tetramethyluronium bromide

TEA triethylamine

TFA trifluoroacetic acid

THF tetrahydrofuran

TLC thin layer chromatography

t_{R} retention time

Reactions are routinely performed under an inert atmosphere such as N₂ gas in air dried glassware. Solvents are used as received from the vendor. Evaporations are performed in a rotary evaporator at reduced pressure and a water bath temperature of 50° C. LC-MS characterizations are performed on a Finnigan HP1100 platform using ESI, and positive ion detection with a Navigator AQK detector. Analytical liquid chromatographic separations are performed by Method A, or where indicated, by Method B. Method A consists of a C18 column of 30×4.6 mm dimensions and a mobile phase consisting of a 1 minute gradient of 2-95% CH₃CN (containing 0.013 TFA) in water (containing 0.04% TFA) at a flow rate of 0.45 mL/min. Method B consists of a C18 column of 30×4.6 mm dimensions and an isocratic mobile phase consisting of CH₃CN-water (1:9) containing 1% formic acid. Retention time (t_{R}) is given in min. TLC is performed on pre-coated silica gel 60 F₂₅₄ glass-backed plates (Merck). Preparative HPLC is performed on a Varian/Gilson platform using a C18 column of 60×21 mm dimensions and a mobile phase consisting of a gradient of 2 to 95% CH₃CN in water containing 0.05% formic acid.

### Example 1

### 1-[2-(4-Benzenesulfonyl-piperazin-1-yl)-ethyl]-3-(2-methyl-quinolin-4-yl)-urea

### 1A. 1-(2-Chloro-ethyl)-3-(2-methyl-quinolin-4-yl)-urea

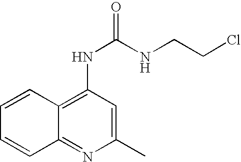

To a solution of 4-amino-2-methylquinoline (12.6 g, 80 mmol) in THF (480 mL) is added 2-chloroethylisocyanate (10.2 mL, 120 mmol) at rt. The reaction mixture is stirred for 40 h at rt. MeOH (100 mL) is added, and stirring is continued an additional hour. The reaction mixture is evaporated and the residue is taken up in CH₂Cl₂. The organic layer is shaken with 1 N HCl (250 mL), and the resulting precipitate is collected by filtration. The solid is washed with CH₂Cl₂ (100 mL), saturated NaHCO₃ (2×100 mL), and with water (4×100 mL). The resulting solid is dried under HV at rt for 14 h to provide the title compound.

### 1B. 4-{2-[3-(2-Methyl-quinolin-4-yl)-ureido]-ethyl}-piperazine-1-carboxylic acid tert-butyl ester

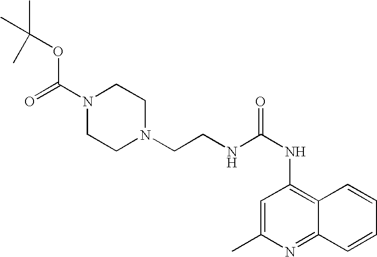

A mixture of piperazine-1-carboxylic acid tert-butyl ester (10 mmol), 1-(2-chloro-ethyl)-3-(2-methyl-quinolin-4-yl)-urea (10 mmol), NaHCO₃ (20 mmol), NaI (0.5 mmol), and THF (70 mL) is stirred in a sealed vessel at 70° C. for 6 d. The reaction mixture is filtered, evaporated to dryness, and the residue is purified by preparative HPLC to provide the title compound.

### 1C. 1-(2-Methyl-quinolin-4-yl)-3-(2-piperazin-1-yl-ethyl)-urea

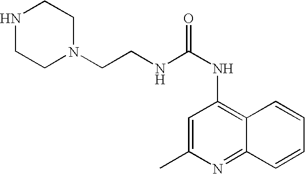

A solution of 4-{2-[3-(2-methyl-quinolin-4-yl)-ureido]-ethyl}-piperazine-1-carboxylic acid tert-butyl ester (5.5 mmol) in AcOH (35 mL) is treated with conc. HCl (3.5 mL). After 30 min, the reaction mixture is frozen and lyophylized to provide the title compound as the dihydrochloride salt.

### 1D. 1-[2-(4-Benzenesulfonyl-piperazin-1-yl)-ethyl]-3-(2-methyl-quinolin-4-yl)-urea

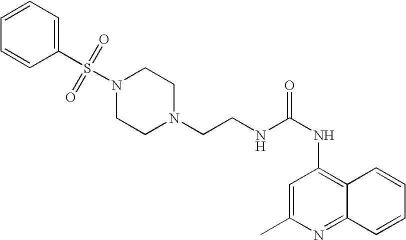

A solution of benzenesulfonyl chloride (0.03 mmol) in THF (1 mL) is added to a mixture of 1-(2-methyl-quinolin-4-yl)-3-(2-piperazin-1-yl-ethyl)-urea (0.025 mmol), DIPEA (6 mL) and THF (1 mL). The reaction mixture is heated at 40° C. for 18 h, and then is evaporated to dryness. The residue is taken up in formic acid (1 mL), and purified by preparative HPLC to provide the title compound.

The following compounds are prepared analogously:

| | Example | tᵣ | MS (ES+) |
|---|---|---|---|
| 1. | 1-[2-(4-Benzenesulfonyl-piperazin-1-yl)-ethyl]-3-(2- | 0.96 | 454.19 |
| | methyl-quinolin-4-yl)-urea | | |
| 2. | 1-(2-Methyl-quinolin-4-yl)-3-{2-[4-(thiophene-2- | 0.94 | 460.13 |
| | sulfonyl)-piperazin-1-yl]-ethyl}-urea | | |
| 3. | 1-(2-Methyl-quinolin-4-yl)-3-{2-[4-(toluene-3-sulfonyl)- | 1.05 | 468.22 |
| | piperazin-1-yl]-ethyl}-urea | | |
| 4. | 1-(2-Methyl-quinolin-4-yl)-3-{2-[4-(toluene-2-sulfonyl)-piperazin- | 1.03 | 468.18 |
| | 1-yl]-ethyl}-urea | | |
| 5. | 1-{2-[4-(2-Fluoro-benzenesulfonyl)-piperazin-1-yl]- | 0.98 | 472.19 |
| | ethyl}-3-(2-methyl-quinolin-4-yl)-urea | | |
| 6. | 1-{2-[4-(3-Fluoro-benzenesulfonyl)-piperazin-1-yl]- | 1.03 | 472.18 |
| | ethyl}-3-(2-methyl-quinolin-4-yl)-urea | | |
| 7. | 1-{2-[4-(4-Fluoro-benzenesulfonyl)-piperazin-1-yl]- | 1.01 | 472.17 |
| | ethyl}-3-(2-methyl-quinolin-4-yl)-urea | | |
| 8. | 1-{2-[4-(3-Cyano-benzenesulfonyl)-piperazin-1-yl]- | 0.99 | 479.17 |
| | ethyl}-3-(2-methyl-quinolin-4-yl)-urea | | |
| 9. | 1-{2-[4-(4-Cyano-benzenesulfonyl)-piperazin-1-yl]- | 1.00 | 479.21 |
| | ethyl}-3-(2-methyl-quinolin-4-yl)-urea | | |
| 10. | 1-{2-[4-(3-Methoxy-benzenesulfonyl)-piperazin-1-yl]- | 1.04 | 484.2 |
| | ethyl}-3-(2-methyl-quinolin-4-yl)-urea | | |
| 11. | 1-{2-[4-(3-Chloro-benzenesulfonyl)-piperazin-1-yl]- | 1.09 | 488.16 |
| | ethyl}-3-(2-methyl-quinolin-4-yl)-urea | | |
| 12. | 1-{2-[4-(4-Chloro-benzenesulfonyl)-piperazin-1-yl]- | 1.10 | 488.14 |
| | ethyl}-3-(2-methyl-quinolin-4-yl)-urea | | |
| 13. | 1-{2-[4-(2-Chloro-benzenesulfonyl)-piperazin-1-yl]- | 1.03 | 488.13 |
| | ethyl}-3-(2-methyl-quinolin-4-yl)-urea | | |
| 14. | 3-(4-{2-[3-(2-Methyl-quinolin-4-yl)-ureido]-ethyl}- | 0.81 | 498.16 |
| | piperazine-1-sulfonyl)-benzoic acid | | |
| 15. | 1-(2-Methyl-quinolin-4-yl)-3-{2-[4-(naphthalene-2- | 1.04 | 504.18 |
| | sulfonyl)-piperazin-1-yl]-ethyl}-urea | | |
| 16. | 1-(2-Methyl-quinolin-4-yl)-3-{2-[4-(naphthalene-1- | 1.03 | 504.16 |
| | sulfonyl)-piperazin-1-yl]-ethyl}-urea | | |
| 17. | 1-(2-Methyl-quinolin-4-yl)-3-{2-[4-(quinoline-8- | 0.87 | 505.16 |
| | sulfonyl)-piperazin-1-yl]-ethyl}-urea | | |
| 18. | 1-(2-Methyl-quinolin-4-yl)-3-{2-[4-(4-trifluoromethyl- | 1.05 | 522.18 |
| | benzenesulfonyl)-piperazin-1-yl]-ethyl}-urea | | |
| 19. | 1-(2-Methyl-quinolin-4-yl)-3-{2-[4-(2-trifluoromethyl- | 1.00 | 522.15 |
| | benzenesulfonyl)-piperazin-1-yl]-ethyl}-urea | | |
| 20. | 1-(2-Methyl-quinolin-4-yl)-3-{2-[4-(3-trifluoromethyl- | 1.05 | 522.14 |
| | benzenesulfonyl)-piperazin-1-yl]-ethyl}-urea | | |
| 21. | 1-{2-[4-(3,4-Dichloro-benzenesulfonyl)-piperazin-1-yl]- | 1.06 | 522.08 |
| | ethyl}-3-(2-methyl-quinolin-4-yl)-urea | | |
| 22. | 1-{2-[4-(4-Butoxy-benzenesulfonyl)-piperazin-1-yl]- | 1.09 | 526.22 |
| | ethyl}-3-(2-methyl-quinolin-4-yl)-urea | | |
| 23. | 1-{2-[4-(4,5-Dichloro-thiophene-2-sulfonyl)-piperazin- | 1.07 | 528 |
| | 1-yl]-ethyl}-3-(2-methyl-quinolin-4-yl)-urea | | |
| 24. | 1-(2-{4-[4-(3-Chloro-2-cyano-phenoxy)- | 1.09 | 605.14 |
| | benzenesulfonyl]-piperazin-1-yl}-ethyl)-3-(2-methyl- | | |
| | quinolin-4-yl)-urea | | |
| 25. | 1-{2-[4-(2-Methanesulfonyl-benzenesulfonyl)- | 0.82 | 532.12 |
| | piperazin-1-yl]-ethyl}-3-(2-methyl-quinolin-4-yl)-urea | | |
| 26. | N-[4-Methyl-5-(4-{2-[3-(2-methyl-quinolin-4-yl)- | 0.84 | 532.1 |
| | ureido]-ethyl}-piperazine-1-sulfonyl)-thiazol-2-yl]- | | |
| | acetamide | | |
| 27. | 1-{2-[4-(3-Bromo-benzenesulfonyl)-piperazin-1-yl]- | 1.00 | 532.01 |
| | ethyl}-3-(2-methyl-quinolin-4-yl)-urea | | |
| 28. | 1-{2-[4-(4-Bromo-benzenesulfonyl)-piperazin-1-yl]- | 1.01 | 532.02 |
| | ethyl}-3-(2-methyl-quinolin-4-yl)-urea | | |
| 29. | 1-(2-Methyl-quinolin-4-yl)-3-{2-[4-(2-trifluoromethoxy- | 1.04 | 538.11 |
| | benzenesulfonyl)-piperazin-1-yl]-ethyl}-urea | | |
| 30. | 1-(2-Methyl-quinolin-4-yl)-3-{2-[4-(4-trifluoromethoxy- | 1.06 | 538.13 |
| | benzenesulfonyl)-piperazin-1-yl]-ethyl}-urea | | |
| 31. | 1-{2-[4-(5-Dimethylamino-naphthalene-1-sulfonyl)- | 1.02 | 547.2 |
| | piperazin-1-yl]-ethyl}-3-(2-methyl-quinolin-4-yl)-urea | | |
| 32. | 1-{2-[4-(5-Chloro-3-methyl-benzo[b]thiophene-2- | 1.10 | 558.11 |
| | sulfonyl)-piperazin-1-yl]-ethyl}-3-(2-methyl-quinolin-4- | | |
| | yl)-urea | | |
| 33. | 1-{2-[4-(4-Bromo-2-ethyl-benzenesulfonyl)-piperazin- | 1.08 | 560.12 |
| | 1-yl]-ethyl}-3-(2-methyl-quinolin-4-yl)-urea | | |
| 34. | 1-{2-[4-(3,5-Bis-trifluoromethyl-benzenesulfonyl)- | 1.10 | 590.1 |
| | piperazin-1-yl]-ethyl}-3-(2-methyl-quinolin-4-yl)-urea | | |
| 35. | N-[5-(4-{2-[3-(2-Methyl-quinolin-4-yl)-ureido]-ethyl}- | 1.00 | 593.12 |
| | piperazine-1-sulfonyl)-thiophen-2-ylmethyl]- | | |
| | benzamide | | |
| 36. | 1-{2-[4-(4-Benzenesulfonyl-thiophene-2-sulfonyl)- | 1.05 | 600.09 |
| | piperazin-1-yl]-ethyl}-3-(2-methyl-quinolin-4-yl)-urea | | |
| 37. | 1-(2-Methyl-quinolin-4-yl)-3-(2-{4-[2-(2,2,2-trifluoro- | 1.05 | 605.19 |
| | acetyl)-1,2,3,4-tetrahydro-isoquinoline-7-sulfonyl]- | | |
| | piperazin-1-yl}-ethyl)-urea | | |
| 38. | 1-(2-Methyl-quinolin-4-yl)-3-[2-(4- | 0.76 | 468.16 |
| | phenylmethanesulfonyl-piperazin-1-yl)-ethyl]-urea | | |
| 39. | 1-(2-Methyl-quinolin-4-yl)-3-{2-[4-(octane-1-sulfonyl)- | 1.05 | 490.25 |
| | piperazin-1-yl]-ethyl}-urea | | |
| 40. | 1-{2-[4-(7,7-Dimethyl-2-oxo-bicyclo[2.2.1]hept-1- | 0.83 | 528.2 |
| | ylmethanesulfonyl)-piperazin-1-yl]-ethyl}-3-(2-methyl- | | |
| | quinolin-4-yl)-urea | | |

### Example 41

### 4-{2-[3-(2-Methyl-quinolin-4-yl)-ureido]-ethyl}-piperazine-1-carboxylic acid biphenyl-2-ylamide

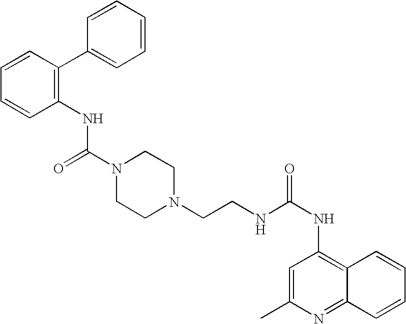

To a solution of 1-(2-methyl-quinolin-4-yl)-3-(2-piperazin-1-yl-ethyl)-urea (0.03 mmol) in THF (0.3 mL) is added a solution of 2-biphenyl isocyanate (0.09 mmol) in THF (0.6 mL). The reaction is allowed to stir for 18 h, and then is quenched with water (0.1 mL), and stirred for an additional 0.5 h. The reaction mixture is evaporated. The residue is taken up in a mixture of formic acid/TFA (1:1; 1 mL), and purified by preparative HPLC.

The following compounds are prepared analogously:

| | | | MS |
|---|---|---|---|
| | Example | tᵣ | (ES+) |
| 41. | 4-{2-[3-(2-Methyl-quinolin-4-yl)-ureido]-ethyl}- | 0.98 | 509.22 |
| | piperazine-1-carboxylic acid biphenyl-2-ylamide | | |
| 42. | 4-{2-[3-(2-Methyl-quinolin-4-yl)-ureido]-ethyl}- | 1.06 | 525.23 |
| | piperazine-1-carboxylic acid (4-phenoxy-phenyl)- | | |
| | amide | | |
| 43. | 4-{2-[3-(2-Methyl-quinolin-4-yl)-ureido]-ethyl}- | 0.81 | 439.24 |
| | piperazine-1-carboxylic acid cyclohexylamide | | |
| 44. | 4-{2-[3-(2-Methyl-quinolin-4-yl)-ureido]-ethyl}- | 0.86 | 447.23 |
| | piperazine-1-carboxylic acid m-tolylamide | | |
| 45. | 4-{2-[3-(2-Methyl-quinolin-4-yl)-ureido]-ethyl}- | 0.78 | 463.19 |
| | piperazine-1-carboxylic acid (4-methoxy-phenyl)- | | |
| | amide | | |
| 46. | 4-{2-[3-(2-Methyl-quinolin-4-yl)-ureido]-ethyl}- | 0.79 | 463.21 |
| | piperazine-1-carboxylic acid (2-methoxy-phenyl)- | | |
| | amide | | |
| 47. | 4-{2-[3-(2-Methyl-quinolin-4-yl)-ureido]-ethyl}- | 0.78 | 447.22 |
| | piperazine-1-carboxylic acid o-tolylamide | | |
| 48. | 4-{2-[3-(2-Methyl-quinolin-4-yl)-ureido]-ethyl}- | 0.91 | 467.16 |
| | piperazine-1-carboxylic acid (3-chloro-phenyl)- | | |
| | amide | | |
| 49. | 4-{2-[3-(2-Methyl-quinolin-4-yl)-ureido]-ethyl}- | 0.97 | 483.21 |
| | piperazine-1-carboxylic acid naphthalen-2-ylamide | | |
| 50. | 4-{2-[3-(2-Methyl-quinolin-4-yl)-ureido]-ethyl}- | 0.78 | 469.18 |
| | piperazine-1-carboxylic acid (2,4-difluoro-phenyl)- | | |
| | amide | | |
| 51. | 4-{2-[3-(2-Methyl-quinolin-4-yl)-ureido]-ethyl}- | 0.73 | 413.2 |
| | piperazine-1-carboxylic acid butylamide | | |
| 52. | 4-{2-[3-(2-Methyl-quinolin-4-yl)-ureido]-ethyl}- | 0.8 | 447.18 |
| | piperazine-1-carboxylic acid benzylamide | | |
| 53. | 4-{2-[3-(2-Methyl-quinolin-4-yl)-ureido]-ethyl}- | 0.76 | 451.21 |
| | piperazine-1-carboxylic acid (2-fluoro-phenyl)- | | |
| | amide | | |
| 54. | 4-{2-[3-(2-Methyl-quinolin-4-yl)-ureido]-ethyl}- | 0.82 | 451.19 |
| | piperazine-1-carboxylic acid (4-fluoro-phenyl)- | | |
| | amide | | |
| 55. | 4-{2-[3-(2-Methyl-quinolin-4-yl)-ureido]-ethyl}- | 0.84 | 451.15 |
| | piperazine-1-carboxylic acid (3-fluoro-phenyl)- | | |
| | amide | | |
| 56. | 4-{2-[3-(2-Methyl-quinolin-4-yl)-ureido]-ethyl}- | 0.86 | 461.23 |
| | piperazine-1-carboxylic acid phenethyl- | | |
| | amide | | |
| 57. | 4-{2-[3-(2-Methyl-quinolin-4-yl)-ureido]-ethyl}- | 0.82 | 463.2 |
| | piperazine-1-carboxylic acid (3-methoxy-phenyl)- | | |
| | amide | | |
| 58. | 4-{2-[3-(2-Methyl-quinolin-4-yl)-ureido]-ethyl}- | 0.84 | 465.21 |
| | piperazine-1-carboxylic acid 4-fluoro-benzylamide | | |
| 59. | 4-{2-[3-(2-Methyl-quinolin-4-yl)-ureido]-ethyl}- | 0.93 | 475.25 |
| | piperazine-1-carboxylic acid (2-isopropyl-phenyl)- | | |
| | amide | | |
| 60. | 4-{2-[3-(2-Methyl-quinolin-4-yl)-ureido]-ethyl}- | 0.96 | 511.1 |
| | piperazine-1-carboxylic acid (4-bromo-phenyl)- | | |
| | amide | | |
| 61. | 4-{2-[3-(2-Methyl-quinolin-4-yl)-ureido]-ethyl}- | 1.02 | 525.2 |
| | piperazine-1-carboxylic acid (2-phenoxy-phenyl)- | | |
| | amide | | |
| 62. | 4-{2-[3-(2-Methyl-quinolin-4-yl)-ureido]-ethyl}- | 0.93 | 467.13 |
| | piperazine-1-carboxylic acid (4-chloro-phenyl)- | | |
| | amide | | |

### Example 63

### 1-(2-{4-[2-(4-Chloro-phenyl)-acetyl]-piperazin-1-yl}-ethyl)-3-(2-methyl-quinolin-4-yl)-urea

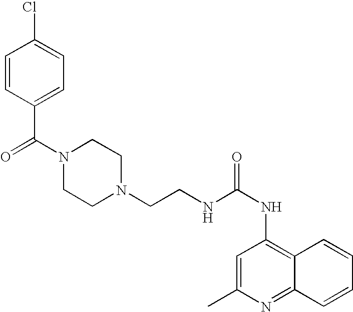

To a solution of 1-(2-methyl-quinolin-4-yl)-3-(2-piperazin-1-yl-ethyl)-urea (0.03 mmol) in DMF (0.3 mL) is added DIPEA (3 eq). The resulting solution is treated with a solution of pyridine-2-carboxylic acid (1.1 eq) in DMF (0.25 mL). A solution of TBTU (1.1 eq) in DMF (0.25 mL) is added. The reaction is stirred at 20 C for 45 min. The reaction mixture is evaporated to dryness. The residue is taken up in CH₃CN/H₂O/TFA (6:10:1; 1 mL) and is purified by preparative HPLC.

The following compounds are prepared analogously:

| | | | MS |
|---|---|---|---|
| | Example | tᵣ | (ES+) |
| 63. | 1-(2-{4-[2-(4-Chloro-phenyl)-acetyl]-piperazin- | 0.92 | 466.17 |
| | 1-yl}-ethyl)-3-(2-methyl-quinolin-4-yl)-urea | | |
| 64. | 1-(2-{4-[2-(4-Methoxy-phenyl)-acetyl]-piperazin- | 0.82 | 462.2 |
| | 1-yl}-ethyl)-3-(2-methyl-quinolin-4-yl)-urea | | |
| 65. | 1-(2-Methyl-quinolin-4-yl)-3-{2-[4-(naphthalene-2- | 0.93 | 468.18 |
| | carbonyl)-piperazin-1-yl]-ethyl}-urea | | |
| 66. | 1-(2-{4-[2-(4-Isopropyl-phenyl)-acetyl]-piperazin- | 1.04 | 474.23 |
| | 1-yl}-ethyl)-3-(2-methyl-quinolin-4-yl)-urea | | |
| 67. | 1-(2-Methyl-quinolin-4-yl)-3-{2-[4-(2-naphthalen- | 0.98 | 482.22 |
| | 1-yl-acetyl)-piperazin-1-yl]-ethyl}-urea | | |
| 68. | 1-[2-(4-Benzoyl-piperazin-1-yl)-ethyl]-3-(2-methyl- | 0.75 | 418.15 |
| | quinolin-4-yl)-urea | | |
| 69. | 1-(2-{4-[3-(4-Fluoro-phenyl)-propionyl]-piperazin- | 0.91 | 464.21 |
| | 1-yl}-ethyl)-3-(2-methyl-quinolin-4-yl)-urea | | |
| 70. | 1-(2-Methyl-quinolin-4-yl)-3-{2-[4-(3-phenyl- | 0.86 | 446.22 |
| | propionyl)-piperazin-1-yl]-ethyl}-urea | | |
| 71. | 1-(2-{4-[2-(4-Fluoro-phenyl)-acetyl]-piperazin- | 0.83 | 450.15 |
| | 1-yl}-ethyl)-3-(2-methyl-quinolin-4-yl)-urea | | |
| 72. | 1-(2-Methyl-quinolin-4-yl)-3-{2-[4-(3-p-tolyl- | 0.95 | 460.25 |
| | propionyl)-piperazin-1-yl]-ethyl}-urea | | |
| 73. | 1-(2-{4-[3-(2-Methoxy-phenyl)-propionyl]- | 0.91 | 476.27 |
| | piperazin-1-yl}-ethyl)-3-(2-methyl- | | |
| | quinolin-4-yl)-urea | | |
| 74. | 1-(2-{4-[3-(4-Methoxy-phenyl)-propionyl]- | 0.87 | 476.24 |
| | piperazin-1-yl}-ethyl)-3-(2-methyl- | | |
| | quinolin-4-yl)-urea | | |
| 75. | 1-(2-{4-[3-(3-Methoxy-phenyl)-propionyl]- | 0.89 | 476.26 |
| | piperazin-1-yl}-ethyl)-3-(2-methyl- | | |
| | quinolin-4-yl)-urea | | |
| 76. | 1-(2-Methyl-quinolin-4-yl)-3-[2-(4-phenylacetyl- | 0.80 | 432.24 |
| | piperazin-1-yl)-ethyl]-urea | | |
| 77. | 1-(2-Methyl-quinolin-4-yl)-3-{2-[4-(2-m-tolyl- | 0.88 | 446.21 |
| | acetyl)-piperazin-1-yl]-ethyl}-urea | | |
| 78. | 1-(2-Methyl-quinolin-4-yl)-3-{2-[4-(2-p-tolyl- | 0.88 | 446.2 |
| | acetyl)-piperazin-1-yl]-ethyl}-urea | | |
| 79. | 1-{2-[4-(3-Chloro-benzoyl)-piperazin-1-yl]- | 0.85 | 452.2 |
| | ethyl}-3-(2-methyl-quinolin-4-yl)-urea | | |
| 80. | 1-(2-Methyl-quinolin-4-yl)-3-{2-[4-(quinoline-6- | 0.63 | 469.22 |
| | carbonyl)-piperazin-1-yl]-ethyl}-urea | | |
| 81. | 1-{2-[4-(4-tert-Butyl-benzoyl)-piperazin-1-yl]- | 1.04 | 474.26 |
| | ethyl}-3-(2-methyl-quinolin-4-yl)-urea | | |
| 82. | 1-(2-{4-[2-(4-Dimethylamino-phenyl)-acetyl]- | 0.62 | 475.32 |
| | piperazin-1-yl}-ethyl)-3-(2-methyl- | | |
| | quinolin-4-yl)-urea | | |
| 83. | 1-{2-[4-(2,4-Dimethoxy-benzoyl)-piperazin-1- | 0.80 | 478.21 |
| | yl]-ethyl}-3-(2-methyl-quinolin-4-yl)-urea | | |
| 84. | 1-(2-Methyl-quinolin-4-yl)-3-{2-[4-(4-pyrrol- | 0.95 | 483.22 |
| | 1-yl-benzoyl)-piperazin-1-yl]-ethyl}-urea | | |
| 85. | 1-(2-{4-[2-(4-Bromo-phenyl)-acetyl]- | 0.95 | 510.14 |
| | piperazin-1-yl}-ethyl)-3-(2-methyl- | | |
| | quinolin-4-yl)-urea | | |
| 86. | 1-{2-[4-(4-Benzoyl-benzoyl)-piperazin-1-yl]- | 0.98 | 522.18 |
| | ethyl}-3-(2-methyl-quinolin-4-yl)-urea | | |

### Example 87

### 1-[2-(4-Benzyl-piperazin-1-yl)-ethyl]-3-quinolin-4-yl-urea

### 87A. 1-(2-Chloroethyl)-3-quinolin-4-yl-urea

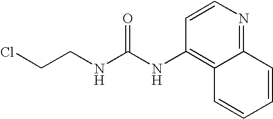

To a solution of 4-aminoquinoline (3.46 g, 24 mmol) in dry THF is added chloroethylisocyanate (3.1 mL, 36 mmol). The reaction mixture is stirred for 18 h. MeOH (10 mL) is added, and stirring is continued for an additional hour. The reaction mixture is evaporated, and partitioned between DCM and aqueous 5% citric acid (150 mL). The aqueous layer is carefully adjusted to pH 9 with solid NaHCO₃. The precipitate is filtered, washed with H₂O (5×20 mL) and Et₂O (2×20 mL), and dried in vacuo at 45° C. to provide the title compound.

### 87B. 1-[2-(4-Benzyl-piperazin-1-yl)-ethyl]-3-quinolin-4-yl-urea

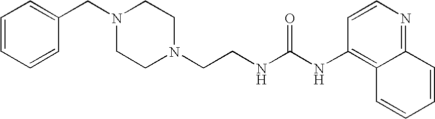

To a solution of 4-benzyl-piperazine (0.03 mmol) in dry THF (1 mL) is added 1-(2-chloro-ethyl)-3-quinolin-4-yl-urea (0.03 mmol), solid NaHCO₃ (2.5 mg), and NaI (1 mg). The flask is tightly sealed, and shaken at 70° C. for 6 days. The reaction mixture is evaporated, taken up in aqueous formic acid, and purified by preparative HPLC to provide the title compound.

The following compounds are prepared in an analogous fashion.

| | | | MS |
|---|---|---|---|
| | Example | tᵣ | (ES+) |
| 87. | 1-[2-(4-Benzyl-piperazin-1-yl)-ethyl]-3- | 0.77 | 390.07 |
| | quinolin-4-yl-urea | | |
| 88. | 1-[2-(4-Benzyl-piperazin-1-yl)-ethyl]-3- | 0.62 | 340.09 |
| | pyridin-4-yl-urea | | |
| 89. | 1-{2-[4-(4-Methoxy-phenyl)-3-methyl- | 0.93 | 420.17 |
| | piperazin-1-yl]-ethyl}-3-quinolin-4-yl-urea | | |
| 90. | 1-{2-[4-(2-Methoxy-phenyl)-piperazin- | 0.89 | 406.17 |
| | 1-yl]-ethyl}-3-quinolin-4-yl-urea | | |
| 91. | 1-{2-[4-(3-Methoxy-phenyl)-piperazin-1- | 0.92 | 406.17 |
| | yl]-ethyl}-3-quinolin-4-yl-urea | | |
| 92. | 1-[2-(4-Phenyl-piperazin-1-yl)-ethyl]-3- | 0.88 | 376.14 |
| | quinolin-4-yl-urea | | |
| 93. | 1-[2-(3-Methyl-4-p-tolyl-piperazin-1-yl)-ethyl]- | 1.01 | 404.15 |
| | 3-quinolin-4-yl-urea | | |
| 94. | 1-Quinolin-4-yl-3-[2-(4-m-tolyl-piperazin- | 0.98 | 390.11 |
| | 1-yl)-ethyl]-urea | | |
| 95. | 1-{2-[4-(4-Fluoro-phenyl)-piperazin-1-yl]- | 0.92 | 394.18 |
| | ethyl}-3-quinolin-4-yl-urea | | |
| 96. | 1-[2-(4-Benzo[1,3]dioxol-5-ylmethyl- | 0.79 | 434.16 |
| | piperazin-1-yl)-ethyl]-3-quinolin-4-yl-urea | | |
| 97. | 1-{2-[4-(2-Fluoro-phenyl)-piperazin-1-yl]- | 0.92 | 394.14 |
| | ethyl}-3-quinolin-4-yl-urea | | |
| 98. | 1-Quinolin-4-yl-3-{2-[4-(3-trifluoromethyl- | 1.09 | 444.12 |
| | phenyl)-piperazin-1-yl]-ethyl}-urea | | |
| 99. | 1-Quinolin-4-yl-3-[2-(4-o-tolyl-piperazin-1- | 1.00 | 390.12 |
| | yl)-ethyl]-urea | | |
| 100. | 1-{2-[4-(3-Chloro-phenyl)-piperazin-1- | 1.03 | 410.1 |
| | yl]-ethyl}-3-quinolin-4-yl-urea | | |
| 101. | 1-[2-(4-Benzhydryl-piperazin-1-yl)-ethyl]- | 1.12 | 466.19 |
| | 3-quinolin-4-yl-urea | | |

### Example 102

### In Vitro Biological Characterization

The inhibitory activity of the compounds of general formula 1 on the actions of urotensin II can be demonstrated using the test procedures described hereinafter:

1) Inhibition of Human [¹²⁵I]-Urotensin II Binding to a Rhabdomyosarcoma Cell Line

Whole cell binding of human [¹²⁵I]-urotensin II is performed using human-derived TE-671 rhabdomyosarcoma cells (Deutsche Sammlung von Mikroorganismen und Zelikulturen, cell line #ACC-263), by methods adapted from a whole cell endothelin binding assay (Breu V et al, In vitro characterization of Ro-46-2005, a novel synthetic non-peptide antagonist of ETA and ETB receptors. FEBS Lett. 1993, 334, 210-214).

The assay is performed in 250 μL Dulbecco's Modified Eagle Medium, pH 7.4 (GIBCO BRL, CatNo 31885-023), including 25 mM HEPES (Fluka, CatNo 05473), 1.0% DMSO (Fluka, CatNo 41644) and 0.5% (w/v) BSA Fraction V (Fluka, CatNo 05473) in polypropylene microtiter plates (Nunc, CatNo 442587). 300,000 suspended cells are incubated with gentle shaking for 4 h at 20° C. with 20 pM human [¹²⁵I]Urotensin II (Anawa Trading SA, Wangen, Switzerland, 2130 Ci/mmol) and increasing concentrations of unlabeled antagonist. Minimum and maximum binding are derived from samples with and without 100 nM unlabelled U-II, respectively. After the 4 h incubation period, the cells are filtered onto GF/C filterplates (Packard, CatNo 6005174). The filter plates are dried, and then 50 μL scintillation cocktail (Packard, MicroScint 20, CatNo 6013621) is added to each well. The filterplates are counted in a microplate counter (Packard Bioscience, TopCount NXT).

All test compounds are dissolved and diluted in 100% DMSO. A ten-fold dilution into assay buffer is performed prior to addition to the assay. The final concentration of DMSO in the assay is 1.0%, which is found not to interfere with the binding. IC50 values are defined as the concentration of antagonist inhibiting 50% of the specific binding of [¹²⁵I]human U-II. Specific binding is the difference between maximum binding and minimum binding, as described above. An IC₅₀ value of 0.206 nM is found for unlabeled human U-II. The compounds of the invention are found to have IC₅₀ values ranging from 10 to 1000 nM in this assay.

2) Inhibition of Human Urotensin II-Induced Contractions on Isolated Rat Thoracic Aorta:

Adult Wistar rats are anesthetized and exsanguinated. The proximal thoracic descending aorta is excised, dissected and a 3-5 mm ring is isolated. The endothelium is removed by gentle rubbing of the intimal surface. The ring is suspended in a 10 mL isolated organ bath filled with Krebs-Henseleit solution (in mM; NaCl 115, KCl 4.7, MgSO₄ 1.2, KH₂PO₄ 1.5, NaHCO₃ 25, CaCl₂ 2.5, glucose 10) kept at 37° C. and aerated with 95% O₂ and 5% CO₂. Indomethacin (10⁻⁵ M) is added to the Krebs-Henseleit solution to avoid eicosanoid generation. The ring is stretched to a resting tension of 1 g. Changes of isometric force are measured using force transducers (EMKA Technologies SA, Paris, France). Following an equilibration period, the rings are briefly contracted with KCl (60 mM). Cumulative doses of human urotensin II (10⁻¹² M to 10⁻⁶ M) are added after a 10 min incubation with the test compound or its vehicle. Functional antagonism is measured as the inhibition of maximal contraction to urotensin II.

1. A compound of formula 1 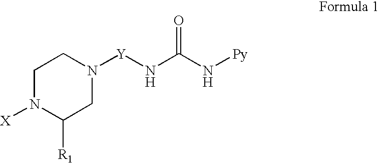
wherein:
Py represents pyridin-4-yl mono-substituted in position 2 with —NR²R³;
pyridin-4-yl di-substituted in position 2 with —NR²R³ and in position 6 with lower alkyl or aryl-lower alkyl; unsubstituted quinolin-4-yl; quinolin-4-yl mono-substituted in position 2 with lower alkyl; or quinolin-4-yl di-substituted in position 2 with lower alkyl and in position 6, 7, or 8 with halogen, lower alkyl, or aryl-lower alkyl;
X represents aryl; aryl-lower alkyl-; lower alkyl disubstituted with aryl-; lower alkyl-SO₂—; aryl-SO₂—; aryl-lower alkyl-SO₂—; lower alkyl-CO—; aryl-CO—; aryl-lower alkyl-CO—; lower alkyl-NR⁶CO—; aryl-NR⁶CO— or aryl-lower alkyl-NR⁶CO—.
Y represents —C(R⁴)(R⁵)—(CH₂)— or —(CH₂)—C(R⁴)(R⁵)—.
R¹ represents hydrogen or a methyl group;
R² and R³ represent independently hydrogen; lower alkyl; or aryl-lower alkyl;
R⁴ represents hydrogen; lower alkyl; aryl; aryl-lower alkyl; or forms together with R⁵ a 3-, 4-, 5-, or 6-membered saturated carbocyclic ring including the carbon atom to which R⁴ and R⁵ are attached as ring atoms;
R⁵ represents hydrogen; methyl; or forms together with R⁴ a 3-, 4-, 5-, or 6-membered saturated carbocyclic ring including the carbon atom to which R⁴ and R⁵ are attached as ring atoms;
R⁶ represents hydrogen; lower alkyl; or aryl-lower alkyl;
or optically pure enantiomers or diastereomers, mixtures of enantiomers or diastereomers, diastereomeric racemates, or mixtures of diastereomeric racemates; or their pharmaceutically acceptable salts, solvent complexes, or morphological forms.

2. The compound of formula 1 according to claim 1, wherein R⁴ and R⁵ represent hydrogen.

3. The compound of formula 1 according to claim 1, wherein R¹ represents hydrogen.

4. The compound of formula 1 according to claim 1, wherein X represents aryl, aryl-lower alkyl- or lower alkyl disubstituted with aryl.

5. The compound of formula 1 according to claim 1, wherein X represents aryl-SO₂— or aryl-lower alkyl-SO₂.

6. The compound of formula 1 according to claim 1, wherein X represents aryl-CO— or aryl-lower alkyl-CO.

7. The compound of formula 1 according to claim 1L wherein X represents aryl-NR⁶CO— or aryl-lower alkyl-NR⁶CO—, and R⁶ has the meaning given in general formula 1.

8. The compound of formula 1 according to claim 1, wherein Py represents unsubstituted quinolin-4-yl or quinolin-4-yl mono-substituted in position 2 with lower alkyl.

9. The compound of formula 1 according to claim 1, wherein Py represents pyridin-4-yl, substituted in position 2 with R²R³N—, wherein R³ represents aryl-lower alkyl and R² represents lower alkyl.

10. The compound of formula 1 according to claim 1, wherein Py represents pyridin-4-yl, substituted in position 2 with R²R³N—, wherein R² represents hydrogen, and R³ has the meaning given in general formula 1.

11. The compound of formula 1 according to claim 1, wherein R⁴ and R⁵ represent hydrogen, and X represents aryl, aryl-lower alkyl- or lower alkyl disubstituted with aryl.

12. The compound of formula 1 according to claim 1, wherein R¹, R⁴ and R⁵ represent hydrogen, and X represents aryl-SO₂— or aryl-lower alkyl-SO₂.

13. The compound of formula 1 according to claim 1, wherein R¹, R⁴ and R⁵ represent hydrogen, and X represents aryl-CO— or aryl-lower alkyl-CO.

14. The compound of formula 1 according to claim 1, wherein R¹, R⁴ and R⁵ represent hydrogen, and X represents aryl-NR⁶CO— or aryl-lower alkyl-NR⁶CO—, and R⁶ has the meaning given in general formula 1.

15. The compound of formula 1 according to claim 1, wherein R¹, R⁴ and R⁵ represent hydrogen, and Py represents unsubstituted quinolin-4-yl or quinolin-4-yl mono-substituted in position 2 with lower alkyl.

16. The compound of formula 1 according to claim 1, wherein R¹, R⁴ and R⁵ represent hydrogen, and Py represents pyridin-4-yl, substituted in position 2 with R²R³N—, wherein R³ represents aryl-lower alkyl and R² represents lower alkyl.

17. The compound of formula 1 according to claim 1, wherein R¹, R⁴ and R⁵ represent hydrogen, and Py represents pyridin-4-yl, substituted in position 2 with R²R³N—, wherein R² represents hydrogen, and R³ has the meaning given in general formula 1.

18. The compound of formula 1 according to claim 1, wherein R¹, R⁴ and R⁵ represent hydrogen, X represents aryl, aryl-lower alkyl- or lower alkyl disubstituted with aryl-, and Py represents unsubstituted quinolin-4-yl or quinolin-4-yl mono-substituted in position 2 with lower alkyl.

19. The compound of formula 1 according to claim 1, wherein R¹, R⁴ and R⁵ represent hydrogen, X represents aryl-SO₂— or aryl-lower alkyl-SO₂—, and Py represents unsubstituted quinolin-4-yl or quinolin-4-yl mono-substituted in position 2 with lower alkyl.

20. The compound of formula 1 according to claim 1, wherein R¹, R⁴ and R⁵ represent hydrogen, X represents aryl-CO— or aryl-lower alkyl-CO—, and Py represents unsubstituted quinolin-4-yl or quinolin-4-yl mono-substituted in position 2 with lower alkyl.

21. The compound of formula 1 according to claim 1, wherein R¹, R⁴ and R⁵ represent hydrogen, X represents aryl-NR⁶CO— or aryl-lower alkyl-NR⁶CO—, and Py represents unsubstituted quinolin-4-yl or quinolin-4-yl mono-substituted in position 2 with lower alkyl, and R⁶ has the meaning given in general formula 1.

22. The compound according to claim 1 selected from the group consisting of:
1-[2-(4-Benzenesulfonyl-piperazin-1-yl)-ethyl]-3-(2-methyl-quinolin-4-yl)-urea;
1-(2-Methyl-quinolin-4-yl)-3-{2-[4-(thiophene-2-sulfonyl)-piperazin-1-yl]-ethyl}-urea;
1-(2-Methyl-quinolin-4-yl)-3-{2-[4-(toluene-3-sulfonyl)-piperazin-1-yl]-ethyl}-urea;
1-(2-Methyl-quinolin-4-yl)-3-{2-[4-(toluene-2-sulfonyl)-piperazin-1-yl]-ethyl}-urea;
1-{2-[4-(2-Fluoro-benzenesulfonyl)-piperazin-1-yl]-ethyl}-3-(2-methyl-quinolin-4-yl)-urea;
1-{2-[4-(3-Fluoro-benzenesulfonyl)-piperazin-1-yl]-ethyl}-3-(2-methyl-quinolin-4-yl)-urea;
1-{2-[4-(4-Fluoro-benzenesulfonyl)-piperazin-1-yl]-ethyl}-3-(2-methyl-quinolin-4-yl)-urea;
1-{2-[4-(3-Cyano-benzenesulfonyl)-piperazin-1-yl]-ethyl}-3-(2-methyl-quinolin-4-yl)-urea;
1-{2-[4-(4-Cyano-benzenesulfonyl)-piperazin-1-yl]-ethyl}-3-(2-methyl-quinolin-4-yl)-urea;
1-{2-[4-(3-Methoxy-benzenesulfonyl)-piperazin-1-yl]-ethyl}-3-(2-methyl-quinolin-4-yl)-urea;
1-{2-[4-(3-Chloro-benzenesulfonyl)-piperazin-1-yl]-ethyl}-3-(2-methyl-quinolin-4-yl)-urea;
1-{2-[4-(4-Chloro-benzenesulfonyl)-piperazin-1-yl]-ethyl}-3-(2-methyl-quinolin-4-yl)-urea;
1-{2-[4-(2-Chloro-benzenesulfonyl)-piperazin-1-yl]-ethyl}-3-(2-methyl-quinolin-4-yl)-urea;
3-(4-{2-[3-(2-Methyl-quinolin-4-yl)-ureido]-ethyl}-piperazine-1-sulfonyl)-benzoic acid;
1-(2-Methyl-quinolin-4-yl)-3-{2-[4-(naphthalene-2-sulfonyl)-piperazin-1-yl]-ethyl}-urea;
1-(2-Methyl-quinolin-4-yl)-3-{2-[4-(naphthalene-1-sulfonyl)-piperazin-1-yl]-ethyl}-urea;
1-(2-Methyl-quinolin-4-yl)-3-{2-[4-(quinoline-8-sulfonyl)-piperazin-1-yl]-ethyl}-urea;
1-(2-Methyl-quinolin-4-yl)-3-{2-[4-(4-trifluoromethyl-benzenesulfonyl)-piperazin-1-yl]-ethyl}-urea;
1-(2-Methyl-quinolin-4-yl)-3-{2-[4-(2-trifluoromethyl-benzenesulfonyl)-piperazin-1-yl]-ethyl}-urea;
1-(2-Methyl-quinolin-4-yl)-3-{2-[4-(3-trifluoromethyl-benzenesulfonyl)-piperazin-1-yl]-ethyl}-urea;
1-{2-[4-(3,4-Dichloro-benzenesulfonyl)-piperazin-1-yl]-ethyl}-3-(2-methyl-quinolin-yl)-urea;
1-{2-[4-(4-Butoxy-benzenesulfonyl)-piperazin-1-yl]-ethyl}-3-(2-methyl-quinolin-4-yl)-urea;
1-{2-[4-(4,5-Dichloro-thiophene-2-sulfonyl)-piperazin-1-yl]-ethyl}-3-(2-methyl-quinolin-4-yl)-urea;
1-(2-{4-[4-(3-Chloro-2-cyano-phenoxy)-benzenesulfonyl]-piperazin-1-yl}-ethyl)-3-(2-methyl-quinolin-4-yl)-urea;
1-{2-[4-(2-Methanesulfonyl-benzenesulfonyl)-piperazin-1-yl]-ethyl}-3-(2-methyl-quinolin-4-yl)-urea;
N-[4-Methyl-5-(4-{2-[3-(2-methyl-quinolin-4-yl)-ureido]-ethyl}-piperazine-1-sulfonyl)-thiazol-2-yl]-acetamide;
1-{2-[4-(3-Bromo-benzenesulfonyl)-piperazin-1-yl]-ethyl}-3-(2-methyl-quinolin-4-yl)-ure;
1-{2-[4-(4-Bromo-benzenesulfonyl)-piperazin-1-yl]-ethyl}-3-(2-methyl-quinolin-4-yl)-urea;
1-(2-Methyl-quinolin-4-yl)-3-{2-[4-(2-trifluoromethoxy-benzenesulfonyl)-piperazin-1-yl]-ethyl}-urea;
1-(2-Methyl-quinolin-4-yl)-3-{2-[4-(4-trifluoromethoxy-benzenesulfonyl)-piperazin-1-yl]-ethyl}-urea;
1-{2-[4-(5-Dimethylamino-naphthalene-1-sulfonyl)-piperazin-1-yl]-ethyl}-3-(2-methyl-quinolin-4-yl)-urea;
1-{2-[4-(5-Chloro-3-methyl-benzo[b]thiophene-2-sulfonyl)-piperazin-1-yl]-ethyl}-3-(2-methyl-quinolin-4-yl)-urea;
1-{2-[4-(4-Bromo-2-ethyl-benzenesulfonyl)-piperazin-1-yl]-ethyl}-3-(2-methyl-quinolin-4-yl)-urea;
1-{2-[4-(3,5-Bis-trifluoromethyl-benzenesulfonyl)-piperazin-1-yl]-ethyl}-3-(2-methyl-quinolin-4-yl)-urea;
N-[5-(4-{2-[3-(2-Methyl-quinolin-4-yl)-ureido]-ethyl}-piperazine-1-sulfonyl)-thiophen-2-ylmethyl]-benzamide;
1-{2-[4-(4-Benzenesulfonyl-thiophene-2-sulfonyl)-piperazin-1-yl]-ethyl}-3-(2-methyl-quinolin-4-yl)-urea;
1-(2-Methyl-quinolin-4-yl)-3-(2-{4-[2-(2,2,2-trifluoro-acetyl)-1,2,3,4-tetrahydro-isoquinoline-7-sulfonyl]-piperazin-1-yl}-ethyl)-urea;
1-(2-Methyl-quinolin-4-yl)-3-[2-(4-phenylmethanesulfonyl-piperazin-1-yl)-ethyl]-urea;
1-(2-Methyl-quinolin-4-yl)-3-{2-[4-(octane-1-sulfonyl)-piperazin-1-yl]-ethyl}-urea;
1-{2-[4-(7,7-Dimethyl-2-oxo-bicyclo[2.2.1]hept-1-ylmethanesulfonyl)-piperazin-1-yl]-ethyl}-3-(2-methyl-quinolin-4-yl)-urea;
4-{2-[3-(2-Methyl-quinolin-4-yl)-ureido]-ethyl}-piperazine-1-carboxylic acid biphenyl-2-ylamide;
4-{2-[3-(2-Methyl-quinolin-4-yl)-ureido]-ethyl}-piperazine-1-carboxylic acid (4-phenoxy-phenyl)-amide;
4-{2-[3-(2-Methyl-quinolin-4-yl)-ureido]-ethyl}-piperazine-1-carboxylic acid cyclohexylamide;
4-{2-[3-(2-Methyl-quinolin-4-yl)-ureido]-ethyl}-piperazine-1-carboxylic acid m-tolylamide;
4-{2-[3-(2-Methyl-quinolin-4-yl)-ureido]-ethyl}-piperazine-1-carboxylic acid (4-methoxy-phenyl)-amide;
4-{2-[3-(2-Methyl-quinolin-4-yl)-ureido]-ethyl}-piperazine-1-carboxylic acid (2-methoxy-phenyl)-amide;
4-{2-[3-(2-Methyl-quinolin-4-yl)-ureido]-ethyl}-piperazine-1-carboxylic acid o-tolylamide;
4-{2-[3-(2-Methyl-quinolin-4-yl)-ureido]-ethyl}-piperazine-1-carboxylic acid (3-chloro-phenyl)-amide;
4-{2-[3-(2-Methyl-quinolin-4-yl)-ureido]-ethyl}-piperazine-1-carboxylic acid naphthalen-2-ylamide;
4-{2-[3-(2-Methyl-quinolin-4-yl)-ureido]-ethyl}-piperazine-1-carboxylic acid (2,4-difluoro-phenyl)-amide;
4-{2-[3-(2-Methyl-quinolin-4-yl)-ureido]-ethyl}-piperazine-1-carboxylic acid butylamide;
4-{2-[3-(2-Methyl-quinolin-4-yl)-ureido]-ethyl}-piperazine-1-carboxylic acid benzylamide;
4-(2-[3-(2-Methyl-quinolin-4-yl)-ureido]-ethyl)-piperazine-1-carboxylic acid (2-fluoro-phenyl)-amide;
4-{2-[3-(2-Methyl-quinolin-4-yl)-ureido]-ethyl}-piperazine-1-carboxylic acid (4-fluoro-phenyl)-amide;
4-{2-[3-(2-Methyl-quinolin-4-yl)-ureido]-ethyl}-piperazine-1-carboxylic acid (3-fluoro-phenyl)-amide;
4-{2-[3-(2-Methyl-quinolin-4-yl)-ureido]-ethyl}-piperazine-1-carboxylic acid phenethyl-amide;
4-{2-[3-(2-Methyl-quinolin-4-yl)-ureido]-ethyl}-piperazine-1-carboxylic acid (3-methoxy-phenyl)-amide;
4-{2-[3-(2-Methyl-quinolin-4-yl)-ureido]-ethyl}-piperazine-1-carboxylic acid 4-fluoro-benzylamide;
4-{2-[3-(2-Methyl-quinolin-4-yl)-ureido]-ethyl}-piperazine-1-carboxylic acid (2-isopropyl-phenyl)-amide;
4-{2-[3-(2-Methyl-quinolin-4-yl)-ureido]-ethyl}-piperazine-1-carboxylic acid (4-bromo-phenyl)-amide;
4-{2-[3-(2-Methyl-quinolin-4-yl)-ureido]-ethyl}-piperazine-1-carboxylic acid (2-phenoxy-phenyl)-amide;
4-{2-[3-(2-Methyl-quinolin-4-yl)-ureido]-ethyl}-piperazine-1-carboxylic acid (4-chloro-phenyl)-amide;
1-(2-{4-[2-(4-Chloro-phenyl)-acetyl]-piperazin-1-yl}-ethyl)-3-(2-methyl-quinolin-4-yl)-urea;
1-(2-{4-[2-(4-Methoxy-phenyl)-acetyl]-piperazin-1-yl}-ethyl)-3-(2-methyl-quinolin-4-yl)-urea;
1-(2-Methyl-quinolin-4-yl)-3-{2-[4-(naphthalene-2-carbonyl)-piperazin-1-yl]-ethyl}-urea;
1-(2-{4-[2-(4-Isopropyl-phenyl)-acetyl]-piperazin-1-yl}-ethyl)-3-(2-methyl-quinolin-4-yl)-urea;
1-(2-Methyl-quinolin-4-yl)-3-{2-[4-(2-naphthalen-1-yl-acetyl)-piperazin-1-yl]-ethyl}-urea;
1-[2-(4-Benzoyl-piperazin-1-yl)-ethyl]-3-(2-methyl-quinolin-4-yl)-urea;
1-(2-{4-[3-(4-Fluoro-phenyl)-propionyl]-piperazin-1-yl}-ethyl)-3-(2-methyl-quinolin-4-yl)-urea;
1-(2-Methyl-quinolin-4-yl)-3-{2-[4-(3-phenyl-propionyl)-piperazin-1-yl]-ethyl}-urea;
1-(2-{4-[2-(4-Fluoro-phenyl)-acetyl]-piperazin-1-yl}-ethyl)-3-(2-methyl-quinolin-4-yl)-urea;
1-(2-Methyl-quinolin-4-yl)-3-{2-[4-(3-p-tolyl-propionyl)-piperazin-1-yl]-ethyl}-urea;
1-(2-{4-[3-(2-Methoxy-phenyl)-propionyl]-piperazin-1-yl}-ethyl)-3-(2-methyl-quinolin-4-yl)-urea;
1-(2-{4-[3-(4-Methoxy-phenyl)-propionyl]-piperazin-1-yl}-ethyl)-3-(2-methyl-quinolin-4-yl)-urea;
1-(2-{4-[3-(3-Methoxy-phenyl)-propionyl]-piperazin-1-yl}-ethyl)-3-(2-methyl-quinolin-4-yl)-urea;
1-(2-Methyl-quinolin-4-yl)-3-[2-(4-phenylacetyl-piperazin-1-yl)-ethyl]-urea;
1-(2-Methyl-quinolin-4-yl)-3-{2-[4-(2-m-tolyl-acetyl)-piperazin-1-yl]-ethyl}-urea;
1-(2-Methyl-quinolin-4-yl)-3-{2-[4-(2-p-tolyl-acetyl)-piperazin-1-yl]-ethyl}-urea;
1-{2-[4-(3-Chloro-benzoyl)-piperazin-1-yl]-ethyl}-3-(2-methyl-quinolin-4-yl)-urea;
1-(2-Methyl-quinolin-4-yl)-3-{2-[4-(quinoline-6-carbonyl)-piperazin-1-yl]-ethyl}-urea;
1-{2-[4-(4-tert-Butyl-benzoyl)-piperazin-1-yl]-ethyl}-3-(2-methyl-quinolin-4-yl)-urea;
1-(2-{4-[2-(4-Dimethylamino-phenyl)-acetyl]-piperazin-1-yl}-ethyl)-3-(2-methyl-quinolin-4-yl)-urea;
1-{2-[4-(2,4-Dimethoxy-benzoyl)-piperazin-1-yl]-ethyl}-3-(2-methyl-quinolin-4-yl)-urea;
1-(2-Methyl-quinolin-4-yl)-3-{2-[4-(4-pyrrol-1-yl-benzoyl)-piperazin-1-yl]-ethyl}-urea;
1-(2-{4-[2-(4-Bromo-phenyl)-acetyl]-piperazin-1-yl}-ethyl)-3-(2-methyl-quinolin-4-yl)-urea;
1-{2-[4-(4-Benzoyl-benzoyl)-piperazin-1-yl]-ethyl}-3-(2-methyl-quinolin-4-yl)-urea;
1-[2-(4-Benzyl-piperazin-1-yl)-ethyl]-3-quinolin-4-yl-urea;
1-[2-(4-Benzyl-piperazin-1-yl)-ethyl]-3-pyridin-4-yl-urea;
1-{2-[4-(4-Methoxy-phenyl)-3-methyl-piperazin-1-yl]-ethyl}-3-quinolin-4-yl-urea;
1-{2-[4-(2-Methoxy-phenyl)-piperazin-1-yl]-ethyl}-3-quinolin-4-yl-urea;
1-{2-[4-(3-Methoxy-phenyl)-piperazin-1-yl]-ethyl}-3-quinolin-4-yl-urea;
1-[2-(4-Phenyl-piperazin-1-yl)-ethyl]-3-quinolin-4-yl-urea;
1-[2-(3-Methyl-4-p-tolyl-piperazin-1-yl)-ethyl]-3-quinolin-4-yl-urea;
1-Quinolin-4-yl-3-[2-(4-m-tolyl-piperazin-1-yl)-ethyl]-urea;
1-{2-[4-(4-Fluoro-phenyl)-piperazin-1-yl]-ethyl}-3-quinolin-4-yl-urea;
1-[2-(4-Benzo[1,3]dioxol-5-ylmethyl-piperazin-1-yl)-ethyl]-3-quinolin-4-yl-urea;
1-{2-[4-(2-Fluoro-phenyl)-piperazin-1-yl]-ethyl}-3-quinolin-4-yl-urea;
1-Quinolin-4-yl-3-{2-[4-(3-trifluoromethyl-phenyl)-piperazin-1-yl]-ethyl}-urea;
1-Quinolin-4-yl-3-[2-(4-o-tolyl-piperazin-1-yl)-ethyl]-urea;
1-{2-[4-(3-Chloro-phenyl)-piperazin-1-yl]-ethyl}-3-quinolin-4-yl-urea; and
1-[2-(4-Benzhydryl-piperazin-1-yl)-ethyl]-3-quinolin-4-yl-urea.

23. A pharmaceutical composition comprising a compound of claim 1 and a carrier and/or an adjuvant.

24. (canceled)

25. (canceled)

26. (canceled)

27. A method of treating a patient suffering from a disorder selected from the group consisting of hypertension, atherosclerosis, angina or myocardial ischemia, congestive heart failure, cardiac insufficiency, cardiac arrhythmias, renal ischemia, chronic kidney disease, renal failure, stroke, cerebral vasospasm, cerebral ischemia, dementia, migraine, subarachnoidal hemorrhage, diabetes, diabetic arteriopathy, diabetic nephropathy, connective tissue diseases, cirrhosis, asthma, chronic obstructive pulmonary disease, high-altitude pulmonary edema, Raynaud's syndrome, portal hypertension, thyroid dysfunction, pulmonary edema, pulmonary hypertension, or pulmonary fibrosis, restenosis after balloon or stent angioplasty, cancer, prostatic hypertrophy, erectile dysfunction, hearing loss, amaurosis, chronic bronchitis, asthma, gram negative septicemia, shock, sickle cell anemia, sickle cell acute chest syndrome, glomerulonephritis, renal colic, glaucoma, therapy and prophylaxis of diabetic complications, complications of vascular or cardiac surgery or after organ transplantation, complications of cyclosporin treatment, pain, addiction, schizophrenia, Alzheimer's disease, anxiety, obsessive-compulsive behavior, epileptic seizures, stress, depression, dementias, neuromuscular disorders, and neurodegenerative diseases, said method comprising administering the pharmaceutical composition according to claim 23.

28. The method of claim 27 further comprising administering at least one additional pharmacologically active compound selected from the group consisting of ACE inhibitors, angiotensin II receptor antagonists, endothelin receptor antagonists, vasopressin antagonists, beta-adrenergic antagonists, alpha-adrenergic antagonists, vasopressin antagonists, TNF alpha antagonists, and peroxisome proliferator activator receptor modulators.
